(19) United States
(12) Patent Application Publication
(10) Pub. No.: US 2026/0034206 A1
(43) Pub. Date: Feb. 5, 2026

(54) VACCINE PLATFORM

(71) Applicant: PÉCSI TUDOMÁNYEGYETEM, Pécs (HU)

(72) Inventor: Antal TAPODI, Pécs (HU)

(21) Appl. No.: 18/033,648

(22) PCT Filed: Oct. 26, 2021
(86) PCT No.: PCT/HU2021/050057

§ 371 Date: Apr. 25, 2023

(30) Foreign Application Priority Data
Oct. 26, 2020 (HU) P2000351

(51) Int. Cl. A61K 39/215; A61K 39/00; A61K 39/385; A61P 31/14; C07K 14/47; C12N 7/00

(52) U.S. Cl. CPC A61K 39/215; A61K 39/385; A61P 31/14; C07K 14/47; C12N 7/00; A61K 2039/575; A61K 2039/6031; C12N 2770/20034

(57) ABSTRACT

The invention relates to a vaccine platform, comprising a lipid binding amino acid sequence and an oligomerization sequence. In particular, the lipid binding amino acid sequence and an oligomerization sequence are derived from filensin, a protein with no or minimal immunogenicity. Filensin has an extremely strong membrane binding capacity and oligomerization property, making it an ideal carrier for an antigenic moiety. An immunization platform comprising a nucleic acid sequence(s) coding for a lipid binding amino acid sequence and an oligomerization sequence is also provided.

### FIELD OF THE INVENTION

The invention relates to a novel vaccine platform, comprising a lipid binding amino acid sequence and an oligomerization sequence. In particular, the lipid binding amino acid sequence and an oligomerization sequence are derived from filensin, a protein with no or minimal immunogenicity. Filensin has an extremely strong membrane binding capacity and oligomerisation property, making it an ideal carrier for an antigenic moiety. A novel immunization platform comprising a nucleic acid sequence(s) coding for a lipid binding amino acid sequence and an oligomerization sequence is also provided.

### BACKGROUND OF THE INVENTION

New vaccine platforms are a must in an era of pathogenic agents spreading with unprecedented speed. Filensin is a cytoskeletal protein expressed in the eye lens. Filensin is required for the correct formation of lens intermediate filaments as part of a complex composed of BFSP1, BFSP2 and CRYAA (Tapodi et al. BFSP1 C-terminal domains released by post-translational processing events can alter significantly the calcium regulation of AQP0 water permeability. Experimental Eye Research 185 (2019) 107585). Severe acute respiratory syndrome coronavirus 2 (SARS-CoV-2) is a member of the Coronaviridae family, causing Coronavirus disease 2019 (COVID-19), a life threatening disease. The Spike protein of SARS-CoV-2, which mediates cell entry and membrane fusion, is the principal target of the humoral immune response (Watanabe et al., Science 369, 330-333 (2020) 17 July).

### SHORT DESCRIPTION OF THE INVENTION

The invention relates to a recombinant polypeptide (immunogenic construct) comprising a lipid binding amino acid sequence (LBD), an oligomerization amino acid sequence (OD) and an immunogenic moiety (IM), wherein
(i) the LBD has low immunogenicity or is not immunogenic and has a length of up to 150 amino acids, preferably up to 100 amino acids, preferably up to 85 amino acids or less than 85 amino acids,
(ii) the OD has low immunogenicity or is not immunogenic and has a length of up to 150 amino acids, preferably up to 100 amino acids, preferably up to 85 amino acids or less than 85 amino acids,
(iii) the IM has a length of up to 50 amino acids, up to 40 amino acid, preferably up to 30 amino acids, preferably up to 25 amino acids or less than 25 amino acids or the IM is a nucleic acid sequence encoding a peptide with a length of up to 50 amino acids, up to 40 amino acid, preferably up to 30 amino acids, preferably up to 25 amino acids or less than 25 amino acids.

The invention also relates to an isolated nucleic acid molecule comprising a nucleic acid sequence(s) encoding the recombinant polypeptide (immunogenic construct).

Preferably the recombinant polypeptide (immunogenic construct) or the nucleic acid molecule is for use in a method for eliciting an immune response in a subject. The use of the recombinant polypeptide (immunogenic construct) or the nucleic acid molecule for the preparation of a medicament for eliciting an immune response in a subject is provided.

In another aspect a peptide is provided, the peptide comprising or consisting of a lipid binding amino acid sequence (LBD, lipid binding domain) derived from filensin (BFSP1; beaded filament structural protein 1) or a functional variant or functional fragment thereof, for use in medicine.

In another aspect, an isolated nucleic acid molecule is provided, the nucleic acid molecule comprising or consisting of a nucleic acid sequence coding for a lipid binding amino acid sequence (LBD) derived from filensin (BFSP1), or a functional variant or functional fragment thereof. Preferably the nucleic acid molecule further comprises a nucleic acid sequence coding for an OD. Preferably the nucleic acid molecule is for use in medicine.

Preferably the nucleic acid is DNA, cDNA or RNA, preferably DNA or cDNA comprising one or more modified nucleotids, preferably RNA, preferably mRNA, highly preferably RNA or mRNA comprising one or more modified nucleotids.

Preferably the lipid binding amino acid sequence is a lipid binding amino acid sequence derived from the C-terminus of BFSP1, or a functional variant or functional fragment thereof. Preferably the peptide comprises the amino acid sequence of SEQ ID NO: 1 (hBFSP1 G434-T460) or a functional variant or a functional fragment thereof, preferably the amino acid sequence of SEQ ID NO: 130 or a functional variant or a functional fragment thereof, more preferably the amino acid sequence of SEQ ID NO: 3 (hBFSP1 G434-E463) or a functional variant or a functional fragment thereof. In preferred embodiments the peptide consists of SEQ ID NO: 1 or a functional variant or a functional fragment thereof, preferably the amino acid sequence of SEQ ID NO: 130 or a functional variant or a functional fragment thereof, and more preferably SEQ ID NO: 3 or a functional variant or a functional fragment thereof. Highly preferably the LBD comprises or consists of the amino acid sequence of SEQ ID NO: 3 or a functional variant or a functional fragment thereof.

Preferably the functional variant or a functional fragment is capable of binding to lipids (e.g, an artificial liposome or a cell membrane). Preferably the functional variant or a functional fragment has a sequence that is at least 85%, at least 90% or at least 95% identical with SEQ ID NO: 1, SEQ ID NO: 130 or SEQ ID NO: 3.

Preferably the functional variant or a functional fragment has a sequence that has up to 15 amino up to 10 amino acid or up to 5 amino acid difference from SEQ ID NO: 1, SEQ ID NO: 130 or SEQ ID NO: 3.

Preferably the peptide (in particular the LBD) is attached to an oligomerization amino acid sequence (OD, oligomerization domain). A recombinant polypeptide is provided, comprising the peptide. Preferably the recombinant polypeptide comprises the LBD and an OD. Preferably the recombinant polypeptide comprises the LBD and an IM. Preferably the recombinant polypeptide comprises the LBD and an IM and does not comprise an OD.

In preferred embodiments the oligomerization amino acid sequence is a coiled coil domain, such as a trimeric coiled coil or a pentameric coiled coil.

Preferably the oligomerization amino acid sequence is derived from BFSP1. Preferably the oligomerization amino acid sequence is an oligomerization amino acid sequence derived from the C terminus of BFSP1, or a functional variant or functional fragment thereof. In a preferred embodiment the OD comprises the sequence according to SEQ ID NO: 2 (hBFSP1 L464-P548) or a functional variant or a functional fragment thereof. In yet another preferred embodiment the OD consists of the sequence according to SEQ ID NO: 2 (hBFSP1 L464-P548) or a functional variant or a functional fragment thereof. Preferably the OD comprises or consists of the sequence according to SEQ ID NO: 123 or a functional variant or a functional fragment thereof. Preferably the OD comprises or consists of SEQ ID NO: 124 or a functional variant or a functional fragment thereof.

In another preferred embodiment the OD is of artificial origin. Preferably the OD comprises or consists of an amino acid sequence of SEQ ID NO: 120 or a functional variant or a functional fragment thereof. Preferably the OD comprises or consists of an amino acid sequence of SEQ ID NO: 121 or a functional variant or a functional fragment thereof. Preferably the OD comprises or consists of an amino acid sequence of SEQ ID NO: 125, or a functional variant or a functional fragment thereof. Preferably the functional variant or a functional fragment of the OD is capable of oligomerization (e.g, forming oligomers or polymers). Preferably the functional variant or a functional fragment has a sequence that is at least 85%, at least 90% or at least 95% identical with any one of SEQ ID NO: 2, SEQ ID NO:121-125. Preferably the functional variant or a functional fragment has a sequence that has up to 50 amino acid, up to 45 amino acid, more preferably up to 30 amino acid, more preferably up to 15 amino acid, more preferably up to 10 amino acid or more preferably up to 5 amino acid difference from any one of SEQ ID NO: 2, SEQ ID NO:121-125.

Preferably the OD comprises or consists of an amino acid sequence of SEQ ID NO: 122 or a functional variant or a functional fragment thereof.

In another preferred embodiment the oligomerization amino acid sequence is derived from a protein different from BFSP1, preferably a protein with low or no immunogenicity upon administration to a subject. In a highly preferred embodiment the oligomerization amino acid sequence is derived from COMP. Preferably, the oligomerization amino acid sequence comprises or consists of the sequence shown in SEQ ID NO: 8 or a functional derivative or functional part thereof or the sequence shown in SEQ ID NO: 9 or a functional derivative or functional part thereof. Preferably the functional variant or a functional fragment has a sequence that is at least 85%, at least 90% or at least 95% identical with any one of SEQ ID NO: 2. SEQ ID NO:8-9. Preferably the functional variant or a functional fragment has a sequence that has up to 50 amino acid, up to 45 amino acid, more preferably up to 30 amino acid, more preferably up to 15 amino acid, more preferably up to 10 amino acid or more preferably up to 5 amino acid difference from any one of SEQ ID NO: 2, SEQ ID NO:8-9.

The peptide comprising a lipid binding amino acid sequence derived from BFSP1 or a functional variant or functional fragment thereof may be linked to the OD directly or via a linker (L). Preferably, the linker is a peptide linker, preferably with a length of up to 10, preferably up to 5 amino acids. Highly preferably the linker is GG (-glycine-glycine-).

The nucleic acid molecule preferably comprises sequence(s) coding for the linker(s).

In certain embodiments the (poly)peptide or nucleic acid molecule is for use in a method of eliciting an immune response in a subject. Preferably the (poly)peptide or nucleic acid molecule is for use in a method of treating or preventing an infection by a pathogen. Preferably the (poly)peptide or nucleic acid molecule is for use in a method of treating or preventing cancer.

Preferably the filensin is the human filensin (hBFSP1).

Preferably a hinge region (linker) is attached at the C terminal amino acid of the OD. Preferably the hinge region is up to 10, preferably up to 5 amino acid long. Preferably the hinge region comprises glycine (G). More preferably the hinge region is GG.

Preferably the OD is localized C terminally from the LBD.

In a third aspect a biologically active agent is linked to the peptide directly or via a linker. The bioactive agent may be a lipid, sugar, amino acid molecule, peptide, polypeptide, nucleic acid molecule or drug, e.g, a small molecule drug. The bioactive agent is preferably an immunogenic epitope. The bioactive agent is preferably an anti-cancer agent. The bioactive agent might be a cancer marker or an oncogene.

Preferably the nucleic acid molecule further comprises a nucleic acid sequence coding for a bioactive agent.

Preferably the bioactive agent is an immunogenic agent (IM), preferably an immunogenic amino acid sequence.

Preferably the IM is a nucleic acid sequence coding for an immunogenic amino acid sequence. In certain embodiments more than one IM are present. The more than one IM may be different IMs from the same pathogen or different IMs from different pathogens. The nucleic acid sequence is DNA or RNA, preferably mRNA. In certain embodiments the peptide comprising the LBD from filensin is conjugated to an OD which is conjugated to a biologically active agent, preferably to an IM. The peptide may be linked to the OD directly or via a linker. The OD may be linked to the IM directly or via a linker.

Preferably the length of the immunogenic amino acid sequence is up to 100 amino acids or up to 50 amino acids, preferably up to 25 amino acids, highly preferably 5-15 amino acids or highly preferably 5-25 amino acids.

Preferably the immunogenic amino acid sequence is from SARS-CoV-2, preferably from the spike protein of SARS-CoV-2. In a preferred embodiment the immunogenic amino acid sequence is the C-terminal extended version of the outer loop of the S-protein (P807-D820, SEQ ID NO: 5) or any functional variant or functional fragment thereof, or the N-terminal extended version of the outer loop of the S-protein (F802-R815, SEQ ID NO: 6 or F802-K814, SEQ ID NO: 11) or any functional variant or functional fragment thereof, highly preferably the outer loop (P807-R815, SEQ ID NO: 7 or P807-K814, SEQ ID NO: 10) of the S protein or any functional variant or functional fragment thereof. Preferably the IM comprises any one or more of the sequences listed in Table 1 or any functional variant or functional fragment thereof. Preferably the IM comprises at least one TCE from SARS-CoV-2 or any functional variant or functional fragment thereof. Preferably the IM comprises more than one amino acid sequences or nucleic acid sequences from SARS-CoV-2 or any functional variant or functional fragment thereof. Preferably the functional variant or functional fragment of any one of the sequences from SARS-CoV-2 is capable of eliciting an immune response in a subject. Preferably the functional variant or functional fragment of any one of the sequences from SARS-CoV-2 has a sequence that is up to 8 amino acid or up to 5 amino acid or up to 3 amino acid different from the reference sequence.

The nucleic acid may comprise control sequences, regulating elements or untranslated regions.

Preferably the nucleic acid molecule comprises the sequence according to SEQ ID NO: 126 any functional variant or functional fragment thereof. Preferably the nucleic acid molecule comprises the sequence according to SEQ ID NO: 127 or any functional variant or functional fragment thereof. Preferably the nucleic acid molecule comprises the sequence according to SEQ ID NO 128, or any functional variant or functional fragment thereof. Preferably the nucleic acid molecule comprises the sequence according to SEQ ID NO 129, or any functional variant or functional fragment thereof.

In another aspect a recombinant polypeptide is provided, the polypeptide comprising a lipid binding amino acid sequence (LBD) and an oligomerization amino acid sequence (OD), wherein the polypeptide is linked to an immunogenic moiety (IM), for use in medicine. A nucleic acid molecule is provided, comprising a coding sequence for the recombinant polypeptide

A nucleic acid molecule is provided, comprising a coding sequence for an LBD and an OD. Preferably the nucleic acid molecule is for use in medicine. Preferably the nucleic acid molecule further comprises a coding sequence for an IM. Preferably the immunogenic moiety is an immunogenic amino acid sequence.

The LBD and the OD may be linked via a linker (L).

Preferably the nucleic acid molecule further comprises a coding sequence for one or more linkers.

Preferably, the linker is a peptide linker, preferably with a length of up to 10, preferably up to 5 amino acids.

Optionally and preferably the polypeptide is linked to the immunogenic agent via a linker (L′).

Preferably, the linker is a peptide linker, preferably with a length of up to 10, preferably up to 5 amino acids.

Highly preferably L′ is GG.

Preferably the oligomerization amino acid sequence is a coiled coil, such as a trimeric coiled coil or a pentameric coiled coil.

Preferably, the lipid binding amino acid sequence and the oligomerization amino acid sequence are derived from the same protein, preferably a protein with low or no immunogenicity upon administration to a subject.

Preferably the lipid binding amino acid sequence and the oligomerization amino acid sequence are derived from filensin (BFSP1), more preferably from human filensin (hBFSP1).

Alternatively, the lipid binding amino acid sequence and the oligomerization amino acid sequence are derived from different proteins, preferably proteins with low or no immunogenicity upon administration to a subject.

Preferably the lipid binding amino acid sequence is derived from filensin (BFSP1), more preferably from human filensin (hBFSP1) and the oligomerization amino acid sequence is derived from another protein.

In other preferred embodiments the LBD is derived from PH (Pleckstrin homology domain) superfamily proteins: e.g. ARF (ADP ribosylation factor), PTEN, PKC, IRS1, Dynamin, OPA, Mitofusin, Pleckstrin;
C1-DAG binding superfamily proteins: e.g. PKC, AKAP13;
C2-superfamily proteins: e.g. PTEN, Synaptotagmin, PLC, PLA;
FYVE domain: RhoGEF, EEA1 etc.; PX domain: PLD, PI3K, NOX (NADPH oxidase) etc.; ENTH domain: Epsin, CLINT1 etc.; ANTH domain: HIP1, HIPIR etc.; BAR domain: AMPH, Endophilin etc.; FERM domain: Ezrin, Radixin etc.; PDZ domain: Erbin etc.; Tubby domain: TUB etc.

In a preferred embodiment the oligomerization amino acid sequence is derived from filensin (BFSP1), more preferably from human filensin (hBFSP1).

Preferably the lipid binding amino acid sequence is a lipid binding amino acid sequence derived from the C-terminus of BFSP1, or a functional variant or functional fragment thereof. Preferably the peptide comprises the amino acid sequence of SEQ ID NO: 1 (hBFSP1 G434-T460) or a functional variant or a functional fragment thereof, preferably the amino acid sequence of SEQ ID NO: 130 or a functional variant or a functional fragment thereof, more preferably the amino acid sequence of SEQ ID NO: 3 (hBFSP1 G434-E463) or a functional variant or a functional fragment thereof. In preferred embodiments the peptide consists of SEQ ID NO: 1 or a functional variant or a functional fragment thereof, preferably the amino acid sequence of SEQ ID NO: 130 or a functional variant or a functional fragment thereof, and more preferably SEQ ID NO: 3 or a functional variant or a functional fragment thereof. Highly preferably the LBD comprises or consists of the amino acid sequence of SEQ ID NO: 3 or a functional variant or a functional fragment thereof.

Preferably the functional variant or a functional fragment is capable of binding to lipids (e.g, an artificial liposome or a cell membrane). Preferably the functional variant or a functional fragment has a sequence that is at least 85%, at least 90% or at least 95% identical with SEQ ID NO: 1, SEQ ID NO: 130 or SEQ ID NO: 3.

Preferably the functional variant or a functional fragment has a sequence that has up to 15 amino up to 10 amino acid or up to 5 amino acid difference from SEQ ID NO: 1, SEQ ID NO: 130 or SEQ ID NO: 3.

Preferably the oligomerization amino acid sequence is an oligomerization amino acid sequence derived from the C terminus of BFSP1, or a functional variant or functional fragment thereof. In a preferred embodiment the OD comprises the sequence according to SEQ ID NO: 2 (hBFSP1 L464-P548) or a functional variant or a functional fragment thereof. In yet another preferred embodiment the OD consists of the sequence according to SEQ ID NO: 2 (hBFSP1 L464-P548) or a functional variant or a functional fragment thereof.

Preferably the OD comprises or consists of the sequence according to SEQ ID NO: 123 or SEQ ID NO: 124 or a functional variant or a functional fragment thereof.

Preferably the OD is of artificial origin. Preferably the OD comprises an amino acid sequence of SEQ ID NO: 120 or an amino acid sequence of SEQ ID NO: 12lor an amino acid sequence of SEQ ID NO: 125, or a functional variant or a functional fragment thereof.

Preferably the OD comprises an amino acid sequence of SEQ ID NO: 122 or a functional variant or a functional fragment thereof.

In a preferred embodiment the oligomerization amino acid sequence is derived from the human cartilage oligomeric matrix protein (COMP), also known as thrombospondin-5 protein. Preferably, the oligomerization amino acid sequence is derived from a coiled coil region of COMP. Preferably, the oligomerization amino acid sequence comprises or consists of the sequence shown in SEQ ID NO: 8 or a functional derivative or functional part thereof or the sequence shown in SEQ ID NO: 9 or a functional derivative or functional part thereof.

Preferably the lipid binding amino acid sequence has a length of less than about 100 amino acids, preferably less than about 50 amino acids, preferably less than about 45 amino acids, preferably less than about 40 amino acids, highly preferably less than about 35 amino acids. Preferably the lipid binding amino acid sequence has a length of more than about 5 amino acids.

Preferably the oligomerization amino acid sequence is derived from BFSP1. Preferably the oligomerization amino acid sequence is an oligomerization amino acid sequence derived from the C terminus of BFSP1, or a functional variant or functional fragment thereof. In a preferred embodiment the OD comprises the sequence according to SEQ ID NO: 2 (hBFSP1 L464-P548) or a functional variant or a functional fragment thereof. In yet another preferred embodiment the OD consists of the sequence according to SEQ ID NO: 2 (hBFSP1 L464-P548) or a functional variant or a functional fragment thereof. Preferably the OD comprises or consists of the sequence according to SEQ ID NO: 123 or a functional variant or a functional fragment thereof. Preferably the OD comprises or consists of SEQ ID NO: 124 or a functional variant or a functional fragment thereof.

In another preferred embodiment the OD is of artificial origin. Preferably the OD comprises or consists of an amino acid sequence of SEQ ID NO: 120 or a functional variant or a functional fragment thereof. Preferably the OD comprises or consists of an amino acid sequence of SEQ ID NO: 121 or a functional variant or a functional fragment thereof. Preferably the OD comprises or consists of an amino acid sequence of SEQ ID NO: 125, or a functional variant or a functional fragment thereof.

Preferably the oligomerization amino acid sequence has a length of about 10-200 amino acids, preferably 10-150 amino acids.

Preferably the polypeptide has the structure of: LBD-OD-(L′)-IM or LBD-(L)-OD-(L′)-IM wherein ( ) indicates an optional element. L and L′ are linker moieties and may be the same or different.

In a fourth aspect a pharmaceutical composition is provided, which comprises a lipid vesicle, such as a liposome to which the polypeptide or the peptide is bound.

In another aspect isolated nucleic acid sequences are provided, said nucleic acid sequences coding for any of the polypeptides, peptides, LBDs, ODs or a functional variant or a functional fragment thereof according to any of the aspects. In preferred embodiments the nucleic acid sequence is RNA, e.g, mRNA. In other preferred embodiments the nucleic acid sequence is DNA. The nucleic acid sequence may comprise modified nucleobases.

In any one of the aspects preferably the (poly)peptide is for use in a method of eliciting an immune response in a subject. The immune response is against the immunogenic agent. Preferably the (poly)peptide is for use in a method of immunizing a subject against a pathogenic agent, such as a virus or bacteria. Preferably the pathogen is an RNA virus, preferably a coronavirus, more preferably SARS-CoV-2. The subject is an animal, preferably a mammal, more preferably a human.

In an embodiment a myristoyl group is further attached to the N-terminal end of the polypeptide or the peptide.

In an embodiment myristoylation occurs co-translationally.

Preferably the length of the immunogenic amino acid sequence is up to 100 amino acids or up to 50 amino acids, preferably up to 25 amino acids, highly preferably 5-15 amino acids or highly preferably 5-25 amino acids.

Preferably the immunogenic amino acid sequence is from SARS-CoV-2, preferably from the spike protein of SARS-CoV-2. In a preferred embodiment the immunogenic amino acid sequence is the C-terminal extended version of the outer loop of the S-protein (P807-D820, SEQ ID NO: 5) or any functional variant or functional fragment thereof, or the N-terminal extended version of the outer loop of the S-protein (F802-R815, SEQ ID NO: 6 or F802-K814, SEQ ID NO: 11) or any functional variant or functional fragment thereof, highly preferably the outer loop (P807-R815, SEQ ID NO: 7 or P807-K814, SEQ ID NO: 10) of the S protein or any functional variant or functional fragment thereof. Preferably the IM comprises any one or more of the sequences listed in Table 1. Preferably the IM comprises at least one TCE from SARS-CoV-2. Preferably the IM comprises more than one amino acid sequences or nucleic acid sequences from SARS-CoV-2.

The nucleic acid may comprise control sequences, regulating elements or untranslated regions.

Preferably the nucleic acid molecule comprises the sequence according to SEQ ID NO: 126 any functional variant or functional fragment thereof. Preferably the nucleic acid molecule comprises the sequence according to SEQ ID NO: 127 or any functional variant or functional fragment thereof. Preferably the nucleic acid molecule comprises the sequence according to SEQ ID NO 128, or any functional variant or functional fragment thereof. Preferably the nucleic acid molecule comprises the sequence according to SEQ ID NO 129, or any functional variant or functional fragment thereof.

### BRIEF DESCRIPTION OF THE FIGURES

FIG. 1: (A) Schematic structure of BFSP1: Arrows indicate two of the predicted LBDs of BFSP1. (B) Arrow indicates the LBD derived from the C terminus of BFSP1. D434 and D549 are proteolytic cleavage sites. (C) Amino acid sequence of BFSP1 (SEQ ID NO: 4) --- represents the LBD (lipid binding domain) of BFSP1 fragment (G434-E463). Indicated aspartic acids “D” are the proteolytic cleavage sites D433 and D549. . . , represents the OD (oligomerization domain) of BFSP1 (L464-P548).

FIG. 2: (A) MCF7 cells were transfected with appropriate GFP conjugates of the different BFSP1 fragments including or excluding the predicted LBD. B. MCF7 cells were transfected with appropriate GFP conjugates of the different BFSP1 fragments including or excluding the predicted LBD. Membranes were stained with FM®4-64 membrane stain (red). C. MCF7 cells were transfected with appropriate GFP conjugates of the different BFSP1 fragments including or excluding the predicted LBD. Membranes were stained with FM® 4-64 membrane stain (arrows).

FIG. 3: Overexpression of recombinant G434-P548-GFP fusion peptide in MCF7 cells. G434-P549-GFP peptide binds to the cell membrane and forms large intracellular membrane vesicles.

FIG. 4: A. Nickel affinity column purification of 434-548-His6 tagged fragment of BFSP1. Open arrow represents the monomer. Black arrows represents the oligomers of 434-548-His6 fragment of BFSP1. (Lane 1: Mw, lane 2: bacterial lysate before purification, lanes 3-8 elution fraction of NIC purification.) B: Nickel affinity column purification of Myr-434-548-His6 tagged fragment of BFSP1. Open arrow represents the monomer. Black arrows represents the oligomers of Myr-434-548-His6 fragment of BFSP1. (Lane 1: Mw, lanes 2-3: bacterial lysates before purification, lanes 4-8 elution fraction of NIC purification.)

FIG. 5: The 434-548-His6 fragment of BFSP1 is a substrate for N-myristoyl transferase (NMT). Myristate were biorthogonal labelled by TAMRA-Cy3 fluorescent dye. Open arrow represents the positive control substrate for NMT: Myr-PfARF (ADP ribosylation factor of Plasmodium Falciparum). Black filled arrow represents the Myr-434-548-His6-BFSP1 fragment.

FIG. 6: Purification of 434-548-His6 fragment of BFSP1. A: purified oligomers of 434-548-His6 by Nickel column (NIC), B: Separated oligomers of 434-548-His6 by Size Exclusion Column (SEC), C: Electron microscopic picture of lipid vesicles coated with 434-548-His6 oligomers, D-G: Coomassie staining of SDS-PAGE gels of SEC-fractions of 434-548-His6 oligomers. (1^{st} peak: 32-38, 2^{nd} peak: 69-85, 3^{rd} peak: 86-99)

FIG. 7: Purification of Myr-434-548-His6 fragment of BFSP1: A: purified oligomers of Myr-434-548-His6 by Nickel column (NIC), B: Separated oligomers of Myr-434-548-His6 by Size Exclusion Column (SEC), C: Electron microscopic picture of lipid vesicles coated with Myr-434-548-His6 oligomers, D-H: Coomassie staining of SDS-PAGE gels of SEC-fractions of Myr-434-548-His6 oligomers. (1^{st} peak: 33-54, 2^{nd} peak: 71-87, 3^{rd} peak: 88-99)

FIG. 8: Schematic structure of Myr-434-548-GG-IMMUNE-EPITOPE Platform: A: Predicted structure of liposome stabilized by Myr-434-548 oligomers conjugated with new immune epitope, B: schematic structure of the myristoylated fusion peptide monomer.

FIG. 9: Overexpression of recombinant G434-T460-GFP fusion peptide in MCF7 cells. G434-T460-GFP peptide binds to the cell membrane and forms large intracellular membrane vesicles.

FIG. 10: Membrane binding of TFR123.

FIG. 11: Cloning TFR123-pET28a recombinant expression system. 11A: MCS of pET28a bacterial expression vector. Vector was cut with NcoI-XhoI before insertion. 11B: Schematic figure of PCR amplicon of 5′-NcoI-G434-P548-3′-XhoI. (pET28a-c(+) cloning region: SEQ ID Nos: 86-88, respectively; pET28a-c(+) expression region: SEQ ID Nos: 89-91, respectively.)

FIG. 12. Amino acid sequence of SARS-CoV-2 Spike protein (SEQ ID NO: 12): ---- represents the F802-F823 Conserved unglycosylated sequence. Bold “R” represents the R815 S2′ cleavage site. . . , represents the C-terminal and N-terminal heptad repeated sequences (HRC and HRN).

FIG. 13: Cryo-EM structure of SARS-CoV-2 S glycoprotein: A: 3D structure of amino acid sequence of S2″ cleavage site: F802-F823: FSQILPDPSKPSKRSFIEDLLF (SEQ ID NO: 92). B: 3D structure of the outer loop of the S2′ cleavage site: P807-R815: PDPSKPSKR (SEQ ID NO: 7)

FIG. 14: A. Cloning strategy of TFR123-fusion peptide. B. TFR123 immunisation platform, Elution from NIC with caspase. C. Schematic structure of TFR123-IMMUNE-EPITOPE Platform: a: Predicted structure of liposome stabilized by TFR123 oligomers conjugated with new immune epitope, b: schematic structure of the myristoylated fusion peptide monomer. D. Amino acid sequence of TFR123 platform and CoV-2 epitopes (SEQ ID Nos: 93, 94, 95, 96, respectively).

FIG. 15: A. TFR123 immunisation platform, Elution from NIC with imidazole. B. Schematic structure of TFR123-IMMUNE-EPITOPE Platform: a: Predicted structure of liposome stabilized by TFR123 oligomers conjugated with new immune epitope, b: schematic structure of the myristoylated fusion peptide monomer. C. Amino acid sequence of TFR123 platform and CoV-2 epitopes (SEQ ID Nos: 97, 98, 99, 100, respectively). For PCR primers for TFR123 immune platform conjugated with CoV-2 epitopes see Table 19.

FIG. 16: A. TFR3x3 immunisation platform. B. Schematic structure of Myr-TFR-3x3-CoV-2-EPITOPE Platform: a: Predicted structure of liposome stabilized by Myr-TFR-3x3 oligomers conjugated with new immune epitope, b: schematic structure of the myristoylated fusion peptide monomer. C. Amino acid sequence of TFR3x3 platform and CoV-2 epitopes (SEQ ID Nos: 101-104, respectively). For PCR primers for TFR-3x3 immune platform conjugated with CoV-2 epitopes see Table 20.

FIG. 17: A. TFR-5x5 immunisation platform. B. Schematic structure of Myr-TFR-5x5-CoV-2-EPITOPE Platform: a: Predicted structure of liposome stabilized by Myr-TFR-5x5 oligomers conjugated with new immune epitope, b: schematic structure of the myristoylated fusion peptide monomer. C. Amino acid sequence of TFR5x5 platform and CoV-2 epitopes (SEQ ID Nos: 105-108, respectively). D. For PCR primers for TFR-5x5 immune platform conjugated with CoV-2 epitopes see Table 21.

FIG. 18. Testing polyclonal antibodies generated by the TFR123-S2-C13 vaccine with Western Blot. (A1) Detection of recombinant, purified SARS-CoV-2-S1, -S2 and TVL (Total Virus Lysate, i.e. SARS-CoV-2 infected, inactivated Vero-6 cell lysate in Laemmli buffer) antigenic proteins with the serum of rabbits immunized with TFR123-S2-C13 vaccine. Secondary antibody: anti-Rabbit IgG tagged with peroxidase. (A2):: Detection of recombinant, purified SARS-CoV-2-S1, -S2 and TVL Total Virus Lysate, i.e. SARS-CoV-2 infected, inactivated Vero-6 cell lysate in Laemmli buffer) antigenic proteins with the serum of a human vaccinated with the Pfizer-BioNTech-COVID-19 vaccine. Secondary antibody: anti-Human IgG tagged with peroxidase. (A3): Detection of recombinant, purified SARS-CoV-2-S1,-S2 and TVL (Total Virus Lysate, i.e. SARS-CoV-2 infected, inactivated Vero-6 cell lysate in Laemmli buffer) antigenic proteins with the serum of a human recovered from COVID-19. Secondary antibody: anti-Human IgG tagged with peroxidase. (B) Detection of the recombinant TFR123-S2-C13 antigen with polyclonal rabbit anti-TFR123-S2-C13 antibodies, Western Blot. Primary antibody: polyclonal rabbit anti-TFR123-S2-C13 antibody: 1:200. Secondary antibody: anti-Rabbit-IgG antibody tagged with peroxidise.

FIG. 19: Schematic structure of PCR amplicon. For sequences see Table 3.

FIG. 20: Schematic structure of PCR amplicon. For sequences see Table 4.

FIG. 21: Schematic structure of PCR amplicon. For sequences see Table 5.

FIG. 22: Schematic structure of PCR amplicon. For sequences see Table 6.

FIG. 23: Schematic structure of PCR amplicon. For sequences see Table 7.

FIG. 24: Schematic structure of PCR amplicon. For sequences see Table 8.

FIG. 25: Schematic structure of PCR amplicon. For sequences see Table 9.

FIG. 26: Schematic structure of PCR amplicon. For sequences see Table 10.

FIG. 27: Schematic structure of PCR amplicon. For sequences see Table 11.

FIG. 28: Schematic structure of PCR amplicon. For sequences see Table 12.

FIG. 29: Schematic structure of PCR amplicon. For sequences see Table 13.

FIG. 30: Schematic structure of PCR amplicon. For sequences see Table 14.

FIG. 31: Schematic structure of PCR amplicon. For sequences see Table 15.

FIG. 32: Schematic structure of PCR amplicon. For sequences see Table 16.

FIG. 33: Schematic structure of PCR amplicon. For sequences see Table 17.

FIG. 34: Schematic structure of PCR amplicon. For sequences see Table 18.

FIG. 35: (A) Immunofluorescent image of TFR123 aggregates without hydrophobic modification on the surface of MCF7 cells fixed with PFA. Primary antibody: Rabbit anti-TFR123-antibody. Secondary antibody: anti-Rabbit-IgG-Alexafluor-488 antibody. Red fluorescence (arrows): WGA fluorescent lipid membrane dye. TFR123 and MCF7 interaction 30 min prior to fixation, at 37° C., 5% CO₂ atmosphere, DMEM medium. (B): Immunofluorescent image of TFR123 aggregates with hydrophobic modification on the surface of MCF7 cells fixed with PFA. Primary antibody: Rabbit anti-TFR123-antibody. Secondary antibody: anti-Rabbit-IgG-Alexafluor-488 antibody. Red fluorescence (arrows): WGA fluorescent lipid membrane dye. TFR123 and MCF7 interaction 30 min prior to fixation, at 37° C. 5% CO₂ atmosphere, DMEM medium. (C): “In vivo” confocal fluorescent microscopic image of MCF7 cells transfected with TFR123-pEGFPN3 plasmid, at 37° C., 5% CO₂ atmosphere, DMEM medium.

FIG. 36: TFR123 cloning strategies. (A (SEQ ID NO: 110) C-terminal tagged TFR123: LBD-ODHHHHHH. (B) Myr-His-tag: “MGHHHSHHH” peptide (MetGlyHisHisHisSerHisHisHis; SEQ ID NO: 109) is cloned on the N-terminus of TFR123-Covid peptide (SEQ ID NO 111). (C) Myr-His-tag extra: “MGHHHSKHHH” peptide is cloned on the N-terminus of TFR123-Covid peptide (SEQ ID NO 112). (D (SEQ ID NO 112)) Inter-chain His-tag: The affinity tag interrupts the sequence of TFR123-Covid recombinant peptide: Myr-TFR123-GGHHHHHHGG-Covid (=Myr-LBDOD-GGHHHHHHGG-Covid). (E (SEQ ID NO: 113)) Co- assembly of Myr-TFR123-HHHHHH (A) with HHHHHHTFR123-Covid peptide.

FIG. 37: TFR123 cloning strategies. (A, SEQ ID NO: 115) DNA sequence of TFR123 Myr-His-tag. (B, SEQ ID NO: 116) DNA sequence of TFR123 Myr-His-tag extra. (C, SEQ ID NO: 117): DNA sequence of TFR123 Inter-chain His-tag. (D, SEQ ID NO: 118) DNA sequence of HHHHHH-TFR123. (E, SEQ ID NO: 119) DNA sequence of TFR123-HHHHHH.

FIG. 38: (A) RNA sequence of TFR123 (SEQ ID NO:118). (B) Complete RNA sequence of TFR123 based on WHO data of Pfizer COVID-19 vaccine (WHO Nonproprietary Names Programme 9/2020 11889, accessed on 22 Oct. 2021) (SEQ ID NO 118 and 119)

### DETAILED DESCRIPTION OF THE INVENTION

The immunization platforms described herein are useful for eliciting an immune response to antigenic moieties (e.g, peptides), which are considered too short when using known methods. These antigenic moieties may play an essential role in an infection with a pathogen or in the immune response to a pathogen, but because of technical difficulties, are underrepresented in the immunization techniques currently used. For example, a number of CD8⁺ T cell epitope sequences (which are essential for the differentiation of memory T-cells) were identified in the viral proteins of SARS-CoV-2 (Ferretti, et al (2020) Unbiased Screens Show CD8(+) T Cells of COVID-19 Patients Recognize Shared Epitopes in SARS-CoV-2 that Largely Reside outside the Spike Protein. Immunity, 53: (5) 1095-1107 e3.), among which only a few were located on the S protein (Ferretti et al. Unbiased Screens Show CD8+ T Cells of COVID-19 Patients Recognize Shared Epitopes in SARS-CoV-2 that Largely Reside outside the Spike Protein. Immunity. 2020 Nov. 17;53(5):1095-1107.e3. doi: 10.1016/j.immuni.2020.10.006), the main target of the vaccines developed early against the pathogen. The immunization platforms described herein provide an appropriate immunization surface for very short epitopes, such as the RBM (receptor binding motif), S1S2 cleavage site or TCE-1 (T-cell epitope 1) of SARS-CoV-2 (Mahajan, S.,et al. Immunodominant T-cell epitopes from the SARS-CoV-2 spike antigen reveal robust pre-existing T-cell immunity in unexposed individuals. Sci Rep 11, 13164 (2021)., Saini et al. SARS-CoV-2 genome-wide T cell epitope mapping reveals immunodominance and substantial CD8+ T cell activation in COVID-19 patients. SCIENCE IMMUNOLOGY 14 Apr. 2021Vol 6, Issue 58DOI: 10.1126/sciimmunol.abf7550). Thereby short epitopes can be used as effective antiviral tools and a more precise targeting of pathogens can be achieved.

The term “peptide” refers to molecules comprising amino acids joined covalently by peptide bonds. The term “polypeptide” or “protein” refers to large peptides, but in general the terms “peptide”, “polypeptide” and “protein” are synonyms and are used interchangeably herein. The terms “peptide”. “polypeptide” and “protein” are used in their accepted scientific meaning.

A “nucleic acid” may be DNA or RNA, e.g in vitro transcribed RNA or synthetic RNA. A nucleic acid may be present as a single-stranded or double-stranded and linear or covalently circularly closed molecule. The nucleic acid can be modified e.g, by stabilizing sequences, capping, or polyadenylation.

The term “lipid binding amino acid sequence” refers to an amino acid sequence that is capable to interact with phospholipids, such as membrane lipids. Polypeptides and proteins comprising a lipid binding amino acid sequence are thus capable to interact proteins, with different affinities.

The terms “lipid binding amino acid sequence” and “lipid binding domain” are used interchangeably. Likewise, the terms “oligomerization amino acid sequence” and “oligomerization domain” are also used interchangeably.

“Human filensin” (“hBFSP1” or “BFSP1”) refers to the protein identified as UniProtKB-Q12934 (BFSP1_HUMAN). The amino acid sequence of hBFSP1 used in the description is that of Isoform 1, having the identifier: Q12934-1. Initially, several lipid binding sequences were predicted in the protein. The lipid binding domain derived from the C terminus of BFSP1 refers to the lipid binding motives in the C-terminal fragment (G434-S665) identified by Tapodi et al. (Tapodi et al, supra). The lipid binding domain derived from the C terminus of BFSP1 refers to the motives comprising SEQ ID NO 1 or 3.

“Human cartilage oligomeric matrix protein” (“COMP”) refers to the protein identified as UniProtKB-P49747 (COMP_HUMAN). The N-terminal coiled coil region of COMP is identified in the InterPro database as IPR039081. SEQ ID NO: 8 represents the amino acid sequence of a trimeric coiled coil from COMP and SEQ ID NO: 9 represents a pentameric coiled coil from COMP.

Preferably the terms “(poly)peptide”, “protein”, “amino acid sequence”, “lipid binding domain”, “oligomerization domain”, “nucleic acid molecule”, “nucleic acid sequence”, “immunogenic moiety” includes the functional fragments and variants thereof.

The term “functional fragment” or “functional variant” of an amino acid sequence relates to any fragment or variant exhibiting one or more functional properties identical or similar to those of the amino acid sequence from which it is derived, i.e., it is functionally equivalent.

The term “functional fragment” or “functional variant”, as used herein, in particular refers to a variant sequence that comprises an amino acid sequence that is altered by one or more amino acids compared to the amino acid sequence of the parent sequence and that is capable of fulfilling one or more of the functions of the parent or sequence, e.g., binding to a lipid molecule (i.e, the “functional fragment” or “functional variant” of a lipid binding amino acid sequence derived from a protein) or eliciting an immune response upon administration to a subject (i.e, the “functional fragment” or “functional variant” of an immunogenic sequence derived from a pathogen).

Preferably the functional fragment or variant of the lipid binding amino acid sequence derived from BFSP1 is capable of binding to lipids (e.g, a liposome or cell membrane) and has no or low immunogenicity. Preferably the functional fragment or variant of the lipid binding amino acid sequence derived from BFSP1 comprises a sequence that is at least 85% or at least 90% or at least 95% identical with SEQ ID NO 1 or 3.

Preferably the functional fragment or variant of the OD derived from BFSP1 is capable of oligomerization and has no or low immunogenicity. Preferably the functional fragment or variant of the OD derived from BFSP1comprises a sequence that is at least 85% or at least 90% or at least 95% identical with SEQ ID NO 2.

Preferably the functional fragment or variant of the OD derived from COMP is capable of oligomerization and has no or low immunogenicity. Preferably the functional fragment or variant of the OD derived from COMP comprises a sequence that is at least 85% or at least 90% or at least 95% identical with SEQ ID NO 8 or 9.

Preferably the functional fragment or variant of the OD derived from BFSP1 or COMP is a coiled coil domain, such as a trimeric coiled coil or a pentameric coiled coil.

The term “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. A fragment is usually at least 3 amino acid long.

The term “functional fragment” or “functional variant” of an amino acid sequence or a nucleic acid sequence relates to any fragment or variant exhibiting one or more functional properties identical or similar to those of the amino acid sequence or nucleic acid sequence from which it is derived, i.e., it is functionally equivalent.

The term “functional fragment” or “functional variant”, as used herein, in particular refers to a variant sequence that comprises an amino acid sequence or nucleic acid sequence that is altered by one or more amino acids or nucleotids compared to the parent sequence and that is capable of fulfilling one or more of the functions of the parent sequence, e.g., binding to a lipid molecule, capable of eliciting an immune response or coding an amino acid sequence that is capable of e.g, binding to a lipid molecule or eliciting an immune response.

The term “fragment”, when used in reference to a reference polypeptide or nucleic acid molecule, refers to a polypeptide or nucleic acid molecule in which amino acid residues or nucleotids are deleted as compared to the reference polypeptide or nucleic acid molecule itself, but where the remaining amino acid sequence or nucleic acid sequence is usually identical to the corresponding positions in the reference polypeptide. A fragment is usually at least 3 amino acid long or the length of the fragment of a nucleic acid molecule is at least as long as to encode 3 amino acids.

The term “functional variant” further includes conservatively substituted variants. The term “conservatively substituted variant” refers to a peptide comprising an amino acid residue sequence that differs from a reference peptide by one or more conservative amino acid substitution, and maintains some or all of the activity of the reference peptide as described herein. A “conservative amino acid substitution” is a substitution of an amino acid residue with a functionally similar residue. Examples of conservative substitutions include the substitution of one hydrophobic residue such as isoleucine, valine, leucine or methionine for another and the substitution of one hydrophilic residue for another, such as between arginine and lysine, between glutamine and asparagine, between threonine and serine. The term “conservatively substituted variant” also includes peptides wherein a residue is replaced with a chemically derivatized residue, provided that the resulting peptide maintains some or all of the activity of the reference (poly)peptide as described herein. In some embodiments, the functional variant of a (poly)peptide shares a sequence identity of at least 70%, 75%, 80%, 85%, 90%, 91%, 92%, 93%, 94%, 95%, 96%, 97%, 98% or 99% with the reference peptide. In some embodiments, the functional variant of a (poly)peptide shares a sequence identity of at least 85%, 90%, 95%, or 99% with the reference (poly)peptide.

A functional variant of a reference nucleic acid refers to a nucleic acid encoding the same polypeptide as the reference nucleic acid. In some embodiments, the functional variant of a nucleic acid molecule shares a sequence identity of at least 70%, 75%, 80%, 85%, 90%, 91%, 92%, 93%, 94%, 95%, 96%, 97%, 98% or 99% with the reference nucleic acid molecule. In some embodiments, the functional variant of a nucleic acid molecule shares a sequence identity of at least 85%, 90%, 95%, or 99% with the reference nucleic acid molecule. A functional variant of a reference nucleic acid refers to a nucleic acid encoding the same polypeptide as the reference nucleic acid. A functional variant of a reference nucleic acid may comprise modified nucleotides or nucleosides. A functional variant of a reference nucleic acid molecule may comprise modified nucleotides or nucleosides.

The nucleic acid may be and preferably is a modified nucleic acid molecule. The preparation and use of modified nucleotides and nucleosides are well-known in the art, e.g, from WO2007024708, U.S. Pat. Nos. 10,232,055, 4,373,071, 4,458,066, 5,262,530, 5,700,642, EP3294326, EP1685844, EP1392341, EP2763701, EP2600901, EP1934345, EP1934345, EP3319622, EP2918275, EP3337902. A modified nucleotide contains one or more chemical modifications (e.g., substitutions) in or on the nitrogenous base of the nucleoside. A modified nucleotide can contain chemical modifications in or on the sugar moiety of the nucleoside, or the phosphate. The nucleic acid sequences may comprise one or more modified nucleotides (e.g., pseudouridine, N6-methyladenosine, 5-methylcytidine, 5-methyluridine), preferably pseudouridine.

The nucleic acid sequence may encode a single polypeptide or more peptides linked together in a way that each of the sequences retains its identity (e.g., linked in series) when expressed as an amino acid sequence. The polypeptides generated from the nucleic acid sequence may then be produced as a fusion polypeptide or engineered in such a manner to result in separate polypeptide or peptide sequences that may be linked by linker sequences.

The nucleic acid sequence may typically be an mRNA, having at least one open reading frame that can be translated by a cell. The translation product is a peptide or protein that may act as an antigen (immunogen). In certain embodiments the antigen is a tumor antigen. In those certain embodiments the patient to whom the pharmaceutical (vaccine) composition is to be administered, has a tumor expressing the tumor antigen or is at the risk of developing such tumor.

The term “immunogenic” refers to an agent capable of eliciting an immune response (either cellular or humoral) in a subject. Immunogenic and antigenic may be used interchangeably.

The term “inducing an immune response” also includes “enhancing an immune response”.

The term “TFR123” refers to a recombinant polypeptide comprising the LBD of the human BFSP1 and optionally the OD of the human BFSP1, and is an exemplary embodiment of the immunization platform according to the invention.

“Immunization platform” refers to a (poly)peptide or nucleic acid molecule, which is capable of eliciting an immune response against an antigenic agent linked to the (poly)peptid or nucleic acid sequence. “Immunization platform” also refers to a (poly)peptide or nucleic acid molecule as described herein that is suitable to deliver the antigen (e.g, epitope) to the cells (e.g, immune cells, cells capable of presenting the antigen, cells capaple of expressing the immunogenic amino acid sequence) of the subject to whom the immunization platform is administered. For example, a polypeptide comprising the G434-P548 fragment of BFSP1 is an immunization platform: an antigenic agent (such as an epitope from SARS-CoV-2 or the spike protein of SARS-CoV-2 or any other known antigenic moiety) may be attached to the polypeptide and an immune response to the antigenic agent will be induced in a subject to whom the polypeptide/antigenic agent conjugate is administered. Likewise, a nucleic acid sequence, such as an mRNA sequence coding for e.g, the G434-P548 fragment of BFSP1 is an immunization platform is an immunization platform: a nucleic acid sequence (such as an mRNA sequence) coding for an antigenic agent (such as an epitope from SARS-CoV-2 or the spike protein of SARS-CoV-2 or any other known antigenic moiety) may be attached to the nucleic acid sequence and an immune response to the antigenic agent will be induced in a subject to whom the nucleic acid sequence/nucleic acid sequence coding for the antigenic agent conjugate is administered. The immunization platform accoding to the invention comprises a lipid binding amino acid sequence derived from the C terminus of BFSP1, or a functional variant or functional fragment thereof or a nucleic acid molecule coding for a lipid binding amino acid sequence from the C terminus of BFSP1 or a functional variant or functional fragment thereof. Preferably the immunization platform comprises the amino acid sequence of SEQ ID NO: 1 (hBFSP1 G434-T460) or a functional variant or a functional fragment thereof, more preferably the amino acid sequence of SEQ ID NO: 3 (hBFSP1 G434-E463) or a functional variant or a functional fragment thereof or a nucleic acid molecule coding for said amino acid sequences.

In one embodiment, the peptide sequences are spaced by linkers. The term “linker”, as used herein, relates to a peptide added between two peptide domains such as epitopes or vaccine sequences to connect said peptide domains. It is preferred that the linker sequence reduces steric hindrance between the two peptide domains, is well translated, and supports or allows processing of the epitopes. Furthermore, the linker should have no or only little immunogenic sequence elements. Glycine and/or serine rich linkers are preferred. The linkers each may comprise 1, 2, or more, preferably up to 50 amino acids. Short linkers (e.g, up to 10, up to 9, up to 8, up to 7, up to 6 or up to 5 amino acid long) are preferred.

TFR123 is a recombinant polypeptide that forms a very stable protein polymer on the surface of biological lipid bilayers (cell membrane or liposomes). The N-terminus of the monomer is anchored into the lipid membrane, meanwhile the C-terminus is polymerized into a protein layer on lipid membranes. This protein polymer is capable of displaying any short antigen sequences (e.g. 9-24 amino acids) very stably in an optimal orientation. Adjuvant free immunization of rabbits resulted in an antigen specific immune response against the recombinant, purified TFR123-Covid19epitope fusion protein.

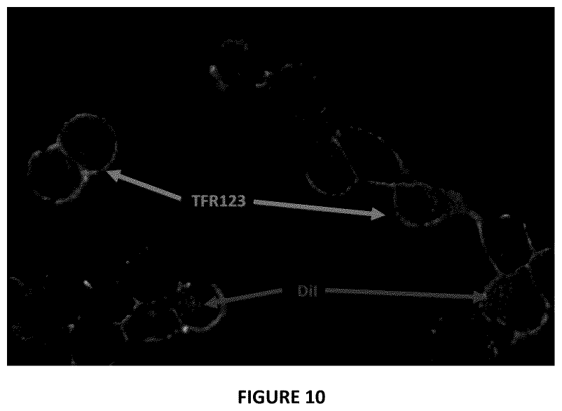

TFR123 (LBD-OD) comprises a LBD (optionally elongated with a linker) and an OD (optionally elongated with a linker). The LBD-OD recombinant polypeptide is elongated on the C-terminus with appropriate antigen sequences (preferably 5-25, or preferably 9-24 amino acids). Purified, recombinant TFR123 monomers polymerize on eukaryotic cell membrane (MCF7) only in ten minutes without penetration (FIG. 10). Transdominantly expressed TFR123-GFP fusion proteins are located in the cell membrane of MCF7 cells and forms large oligomers on the surface of intracellular membrane vesicles (FIG. 3).

Examples of amino acid sequences of preferred LBDs useful in the peptides, polypeptides or immunization platforms described herein are shown in Table 22 in the Examples section.

Examples of amino acid sequences of preferred ODs useful in the peptides, polypeptides or immunization platforms described herein are shown in Table 23 in the Examples section.

### Vaccine Compositions

The immunization platform, e.g. TFR123 may be bound to a lipid vesicle, such as a liposome. Incubation of the liposome-bound myristoylated TFR123-Covepitope (Covepitope refers to an epitope derived from SARS-CoV-2) with mammalian cells showed that the recombinant polypeptide did not cross the cell membrane, rather, it was seen as “beads” binding to the membrane of the mammalian cells (FIG. 10). Thus, the myristoylated polypeptide stabilized the membrane of the liposome while the membrane binding property of the polypeptide is maintained even when bound to the liposome.

A vaccine composition comprising the immunization platform bound to a liposome is advantageous because the epitope can be presented in very high density and in a stable manner.

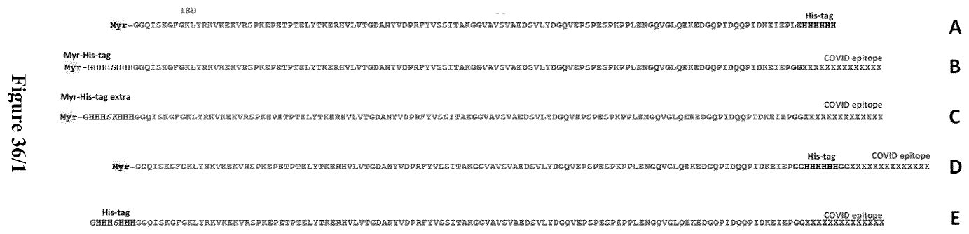

However, TFR123 proved to be highly functional (i.e, capable of binding to the membrane and inducing an appropriate and robust immune response) even without being bound to a liposome (FIG. 35).

Myristoylated TFR 123-Covepitope formed an extremely stable oligomer layer on mammalian cells without the penetration of the polypeptide. Binding to the cell membrane positions the immunogenic epitope on the cell surface with an appropriate exposition and may provide protection against interstitial proteases. Furthermore, unmyristoylated TFR123-Covid-antigen, forming aggregates, also induced antigen selective immune response in rabbits. Skeletal muscle cells could bind and display TFR 123-Covid immunogen.

Without wishing to be bound by theory, the core polymer of the immunizing platform might work as an “internal adjuvant” providing an increased immune response.

In preferred embodiments the vaccine composition comprises a lipid binding amino acid sequence from the C terminus of BFSP1, or a functional variant or functional fragment thereof or a nucleic acid molecule coding for a lipid binding amino acid sequence from the C terminus of BFSP1, or a functional variant or functional fragment thereof. Preferably the immunization platform comprises a peptide comprising the amino acid sequence of SEQ ID NO: 1 (hBFSP1 G434-T460), SEQ ID NO: 3 or SEQ ID NO: 130 or a functional variant or a functional fragment thereof, more preferably the amino acid sequence of SEQ ID NO: 3 (hBFSP1 G434-E463) or a functional variant or a functional fragment thereof or a nucleic acid molecule coding for said peptide and/or amino acid sequences.

The vaccine composition may comprise an adjuvant. “Adjuvant” means any substance that increases the humoral or cellular immune response to an antigen. Adjuvants are well known in the art. Adjuvants comprise a heterogeneous group of compounds such as oil emulsions (e.g. Freund's adjuvants), mineral compounds (such as alum), bacterial products (such as Bordetella pertussis toxin), liposomes, and immune-stimulating complexes. Examples for adjuvants are monophosphoryl-lipid-A, Saponins, vitamin E, montanid, CpG oligonucleotides, and various water-in-oil emulsions which are prepared from biologically degradable oils such as squalene.

The vaccine compositions of the invention can further comprise pharmaceutically acceptable carriers, excipients and/or stabilizers (see e.g. Remington: The Science and practice of Pharmacy (2005) Lippincott Williams).

### Drug Delivery System

TFR123 was shown to form a polymer layer on the surface of artificial liposomes and to stabilize them. Therefore, TFR123 may be used in targeted drug delivery constructs. For example a targeting moiety (such as a specific receptor ligand) is attached to TFR123 instead of an IM to “guide” the construct to the target cell. The construct to which TFR123 is attached is, for example, a liposome carrying an active agent. Likewise, the LBD derived from filensin attached to an OD derived from another protein (such as COMP) can be used instead of TFR123.

A peptide comprising a lipid binding amino acid sequence (LBD) derived from filensin (BFSP1) or a functional variant or functional fragment thereof is provided for use in a drug delivery construct. A polypeptide comprising a lipid binding amino acid sequence (LBD) derived from filensin (BFSP1) or a functional variant or functional fragment thereof and an oligomerization domain is provided for use in a drug delivery construct. In preferred embodiments a targeting moiety (TM) is attached to the peptide or the polypeptide. In preferred embodiments the peptide, the polypeptide or the TM attached to the peptide or the TM attached to the polypeptide is attached to a liposome or a lipid containing construct carrying an active agent.

### mRNA Immunization Platform

In preferred embodiments the vaccine composition comprises nucleic acid sequence(s) coding for the LBD, optionally the OD, and the immunogenic epitope. The protein polymer product of the mRNA vaccine will form a stable antigen-presenting layer on the host cell membrane and on the membrane vesicles (large exosomes) exiting the host cell, leading to a strong immunity elicited by the vaccine.

### Methods of Treatment

A method for the prevention or treatment of an infection or a cancer is provided, wherein the method comprises administering a prophylactically or therapeutically effective amount of an antigen (such as an epitope) linked to an immunization platform described herein (e.g, in the form of a vaccine composition). The antigen may be a nucleic acid sequence coding for an antigen (such as an mRNA sequence coding for an immunogenic amino acid sequence. The prevention or treatment may comprise administering the antigen linked to the immunization platform more than once, e.g, in a primer and booster regime or an annual administration. In particular, a method of preventing, ameliorating or treating a disease caused by an infection with an RNA virus, preferably a coronavirus, highly preferably preventing COVID-19 in a subject is provided, wherein an immunization platform described herein linked to an epitope from SARS-CoV-2 or a nucleic acid sequence encoding an epitope from SARS-CoV-2 is administered to the subject.

### Myristoylation

Myristoilation of the TFR123 platform (or the N-terminal (or C-terminal) myristoilation of a (poly)peptide comprising the lipid binding amino acid sequence (LBD) derived from filensin or a recombinant polypeptide comprising an LBD, an OD and optionally an IM or TM or a recombinant polypeptide comprising an LBD and an IM or TM and not comprising an OD), if desired, may be performed with several different techniques. See a few examples below and in the examples section.

1. Myristoylation in vitro, following elution of the peptide comprising the LBD (and preferably the OD and IM). Elution of the peptide comprising the LBD (and preferably the OD and IM), using a His-DVDP-LBD clone, wherein the DVPD Caspase-3 cleavage site next to the LBD sequence, is performed with His-Caspase-3. Non-cleaved His-DVPD-LBD(-OD-IM) and His-Caspase-3 are removed.

2. MGHHHSHHH-LBD contruct: Candida albicans N-myristyl-transferase (CaNMT) is co-expressed with a MGHHHSHHH-LBD-(OD-IM) construct, therefore myristoylation is performed in the E. coli cells.

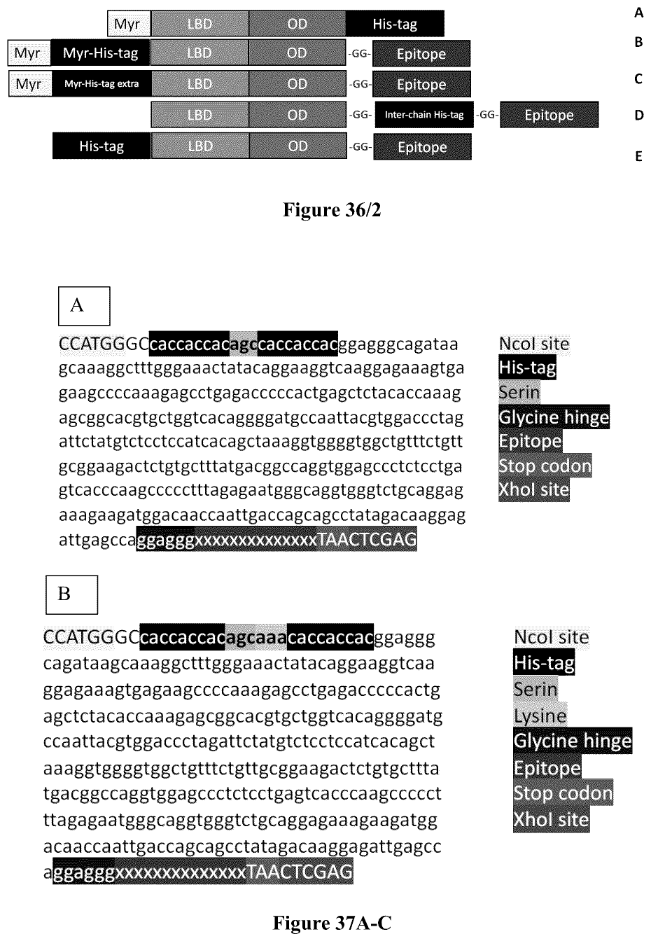

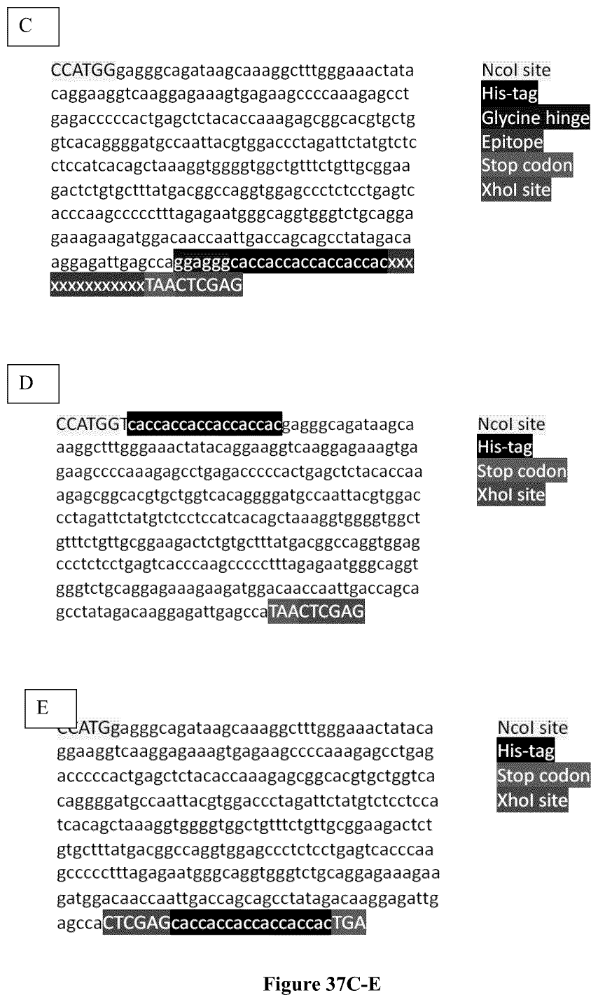

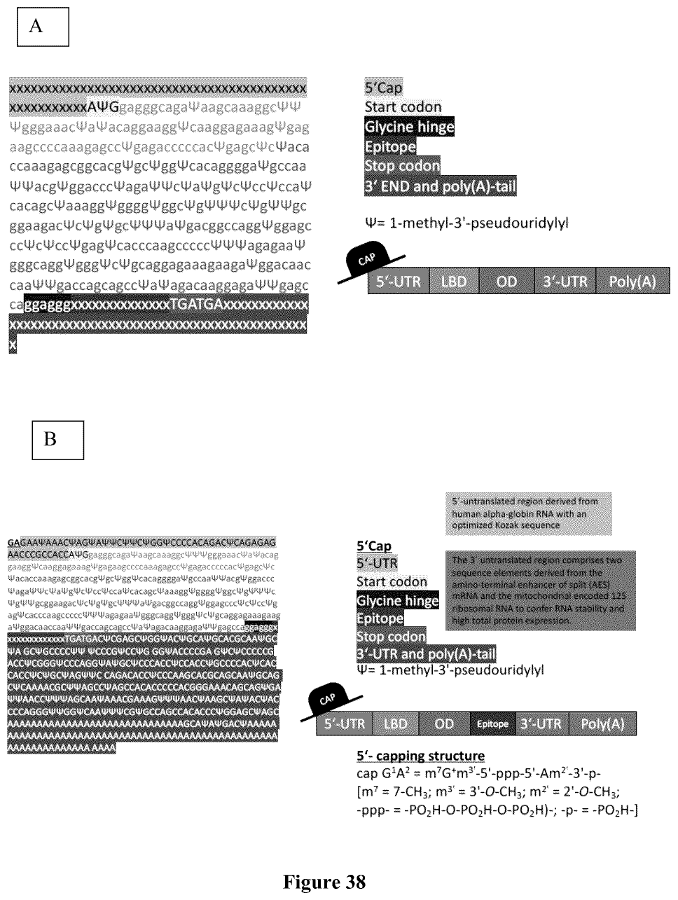

Myr-His-tag: “MGHHHSHHH” peptide (MetGlyHisHisHisSerHisHisHis) is cloned on the N-terminus of TFR123-Covid peptide (FIG. 36B). This Myr-His-tag allows the N-term myrisoylation of glycin as well as purification of new TFR123-Covid recombinant peptides with interrupted His-tag. (Conserved NMT-site: Nmyristoyl-transferase binding site: GxxxSxxx. 1^{st} position Glycine and 5^{th} position Serin, x=any amino acid).

3. MGHHHSKHHH-LBD-(OD-IM) construct or MGHHHSTHHH-LBD: rate of myristoylation may be increased by the insertion of K or T after the serine in position 5. H might be substituted with any amino acid. 1^{st} position is Glycine.

4. Inter-chain His-tag: The affinity tag interrupts the sequence of TFR123-IM recombinant peptide: Myr-TFR123-GGHHHHHHGG-IM (=Myr-LBDOD-GGHHHHHHGG-IM). Affinity tag is located between TFR123 and IM sequence. The flanking double -GG- hinge provides the flexibility of recombinant protein allowing the His-tag to bind to the Nickel Column, while the native myristoylation site is still unmodified on the N-terminus (FIG. 36D).

5. Myr-His-tag extra: “MGHHHSKHHH” peptide is cloned on the N-terminus of TFR123-IM peptide (FIG. 36C). This Myr-His-tag extra allows better N-terminal myrisoylation of TFR123.

6. Co-assembly of Myr-TFR123-HHHHHH (FIG. 36A) with HHHHHHTFR123-(IM) (FIG. 36E): This strategy has several benefits: 1) No cloning of a new construct is needed, 2) Native Myr-site instead of artificial one. 3) His-tag should not be interrupted, 4) Small proportion of myristoylated TFR123 core peptide is required to bind unmyristoylted TFR123-Covid peptide to cell membrane or liposome. The “OD” domain holds together the Myr-LBD-OD-His6 and His6-LBDOD-IM heteromers.

7. Myr-LBD-OD-PP-HHHHHH-PP-IM.

### SARS-CoV-2 Epitopes

The list of epitope sequences of SARS-CoV-2 Spike, that were used for production of TFR123 vaccines. Eight epitopes are related to the Receptor Binding Domain (RBD) of SARS-CoV-2 Spike. Four epitopes close to the TMPRSS2 cleavage site are designed. Three epitopes close to the Furin cleavage site are designed. Six epitopes are designed to the CD8+ T-cell receptor binding sites. (Table 1)

### Table 1 Examples of SARS-CoV-2 Epitopes

| SEQ | Name of | Position in the | |
|---|---|---|---|
| ID NO: | epitope | spike protein | Amino acid sequence |
| 7 | C8N1 | P807-R815 | LPDPSKPSKR |
| 5 | C8N6 | P807-D820 | LPDPSKPSKRSFIED |
| 11 | S2C13 | F802-K814 | FSQILPDPSKPSK |
| 6 | S2C14 | F802-R815 | FSQILPDPSKPSKR |
| 13 | S1S2C14 | A672-R685 | ASYQTQTNSPRRAR |
| 14 | S1S2C24 | C662-R685 | CDIPIGAGICASYQTQTNSPRRAR |
| 15 | RBD-N14 | L461-Q474 | LKPFERDISTEIYQ |
| 16 | RBM-C14 | A475-C488 | AGSTPCNGVEGENC |
| 17 | RBD-C14 | Y489-G502 | YFPLQSYGFQPTNG |
| 18 | RBM-24 | T470-Q493 | TEIYQAGSTPCNGVEGENCYFPLQ |
| 19 | RBD-24 | Y489-V512 | YFPLQSYGFQPTNGVGYQPYRVVV |
| 20 | S2-24 | P792-R815 | PPIKDFGGFNFSQILPDPSKPSKR |
| 21 | NUL-14 | S343-F456 | SKVGGNYNYLYRLF |
| 22 | NUL-24 | V433-F456 | VIAWNSNNLDSKVGGNYNYLYRLF |
| 23 | TCE1-C14 | A264-L277 | AYYVGYLQPRTFLL |
| 24 | TCE2-C14 | S373-K386 | SFSTFKCYGVSPTK |
| 25 | TCE3-C14 | L1203-11216 | LGKYEQYIKWPWYI |
| 26 | TCE1-24 | S254-L277 | SSSGWTAGAAAYYVGYLQPRTFLL |
| 27 | TCE2-24 | A363-K386 | ADYSVLYNSASFSTFKCYGVSPTK |
| 28 | TCE3-24 | L1193-11216 | LNESLIDLQELGKYEQYIKWPWYI |
| 29 | RBM-70 | N437-Q506 | NSNNLDSKVGGNYNYLYRLFRKSN |
| | | | LKPFERDISTEIYQAGSTPCNGVEGF |
| | | | NCYFPLQSYGFQPTNGVGYQ |

A recombinant polypeptide is provided, comprising a lipid binding amino acid sequence (LBD), an oligomerization amino acid sequence (OD) and optionally an immunogenic moiety (IM) or a targeting moiety (TM), wherein
(i) the LBD has low immunogenicity or is not immunogenic and has a length of up to 150 amino acids, preferably up to 100 amino acids, preferably up to 85 amino acids or less than 85 amino acids,
(ii) the OD has low immunogenicity or is not immunogenic and has a length of up to 150 amino acids, preferably up to 100 amino acids, preferably up to 85 amino acids or less than 85 amino acids,
(iii) the IM or the targeting moiety has a length of up to 50 amino acids, up to 40 amino acid, preferably up to 30 amino acids, preferably up to 25 amino acids or less than 25 amino acids or the IM is a nucleic acid sequence encoding a peptide with a length of up to 50 amino acids, up to 40 amino acid, preferably up to 30 amino acids, preferably up to 25 amino acids or less than 25 amino acids.

A nucleic acid molecule comprising a nucleic acid sequence(s) encoding the recombinant polypeptide above is also provided. Preferably the nucleic acid molecule is preferably RNA. Preferably the recombinant polypeptide or the nucleic acid molecule is for use in a method for eliciting an immune response in a subject.

Preferably the recombinant polypeptide is for use in drug delivery. Preferably the recombinant polypeptide is attached to an artificial liposome.

A method for the preparation for the recombinant polypeptide is provided, comprising the recombinant expression of the LBD, the OD and optionally the IM or the TM in a host organization transfected with an expression/transfection vector carrying a coding sequence for the LBD, the OD and the IM, wherein the LBD, the OD and optionally the IM or the TM may be on separate expression/transfection vectors or on the same expression/transfection vector.

### EXAMPLES

### I. TFR123-SARS-CoV-2 Vaccine Procedure

### a. Recombinant Core Peptide which Forms Lipid Binding Oligomers Originated from BFSP1

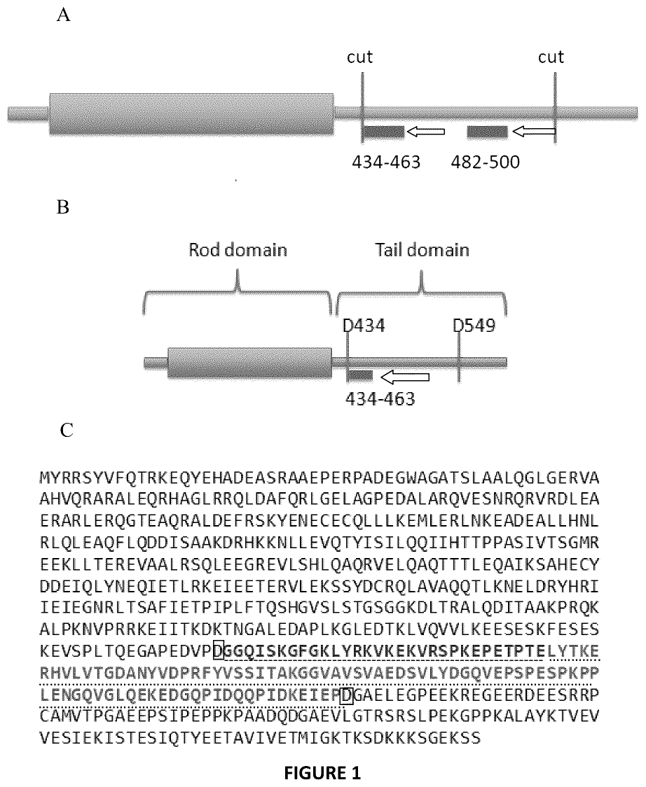

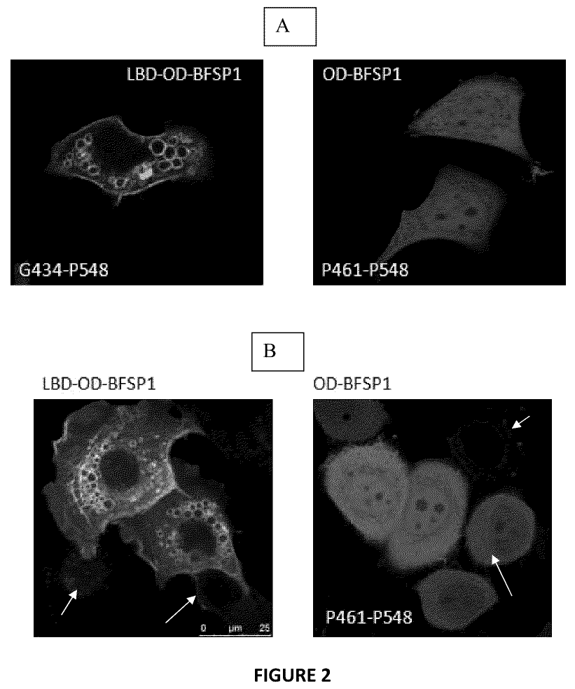

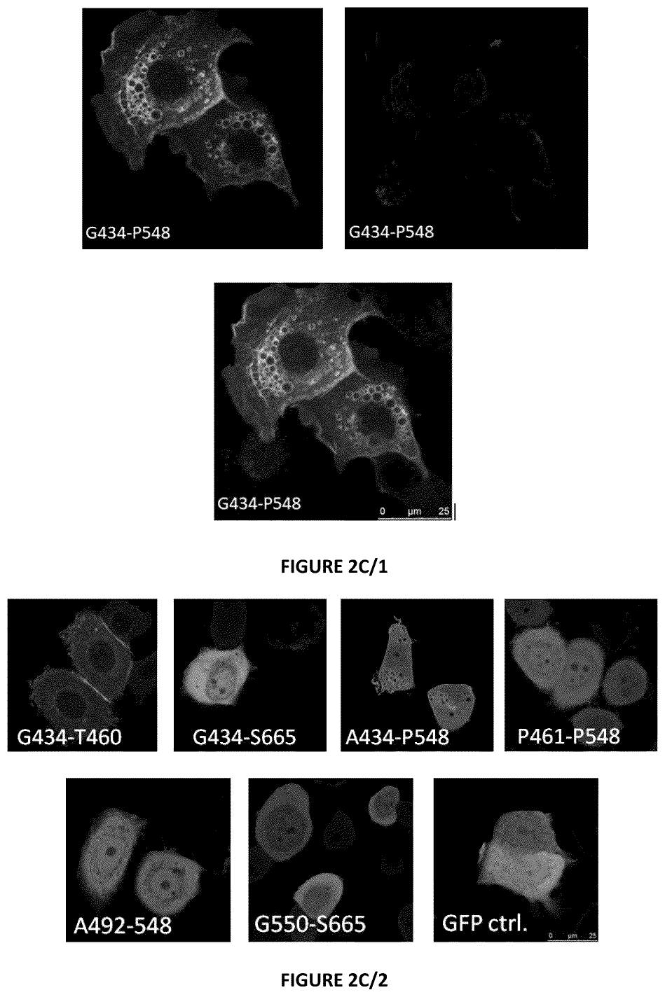

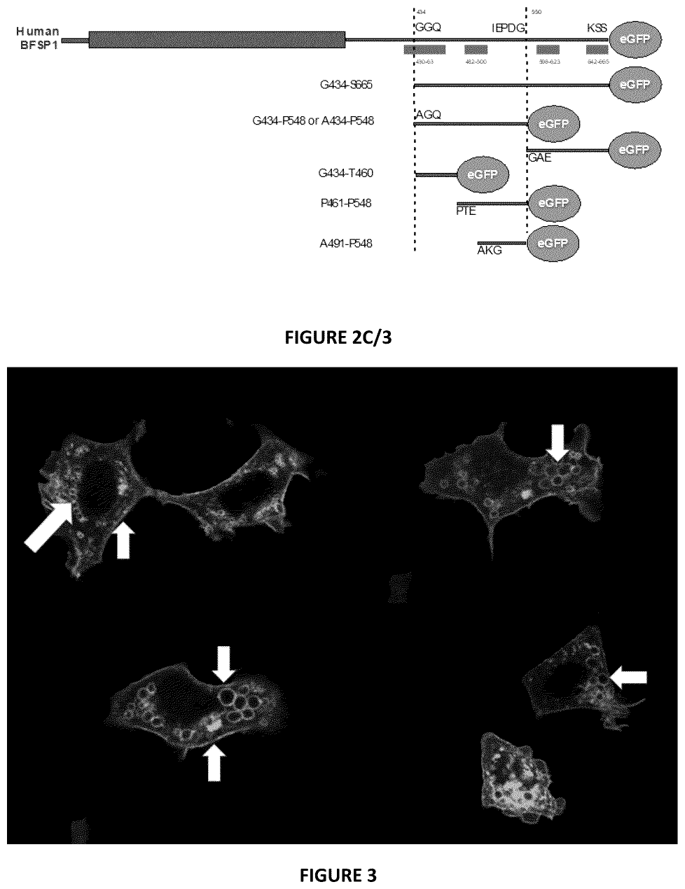

Filensin (BFSP1: Beaded structure protein 1) is a cytoskeletal intermediate filament expressed exclusively in the fibrous cell of eye lens. A very strong lipid binding domain (LBD) was identified during the investigation of endogen proteolysis of BFSP1. A predicted lipid binding motif of BFSP1 is located in the position of G434-E463 amino acid sequence (FIG. 1A and B) (lipid binding amino acid sequence derived from the C terminus of BFSP1). Lipid binding domain (LBD) was proved by overexpression of C-terminal truncated form (P461-P548: OD-BFSP1) of the recombinant proteolytic fragment (G434-P548-GFP: LBD-OD-BFSP1) of BFSP1 fused to GFP in MCF7 cell line. The OD: P461-P548 fragment of BFSP1 excludes the predicted LBD. The C terminal GFP tagged P461-P548 fragment (OD) did not bind to the cell membrane compared to G434-P548-GFP (LBD-OD-BFSP1) (FIG. 2A and B). Membrane binding of additional predicted LBDs (482-500, 598-623 and 642-665) was also excluded (FIG. 2C).

MCF7 cells were seeded on 35 mm glass bottom dish. Cells were transiently transfected with 500 ng pEGFP-N3-BFSP1-fragment eukaryotic expression construct plasmids. Plasmids were preincubated with GeneJuice transfection reagent in OptiMEM medium according to the suggestions of manufacturer. On the next day, cell membranes were stained with FM®4-64 red fluorescent membrane stain. Life cell images were captured in 5% CO2 atmosphere at 37° C. with confocal microscope (Zeiss LSM 710).

### b. G434-P548 Fragment of BFSP1 (“TFR123-Core Peptide”)

To reveal why TFR123 (alias: G434-P548 peptide=LBD-OD-BFSP1) might become a potential immunization platform, it is necessary to understand the oligomerization and lipid binding property of the above-mentioned fragment of BFSP1. Recombinant G434-P548-GFP peptide binds to the cell membrane and forms large intracellular membrane vesicles in MCF7 cells (FIG. 2-3).

MCF7 cells were seeded on 35 mm glass bottom dish. Cells were transiently transfected with 500 ng pEGFP-N3-LBD-OD-BFSP1 eukaryotic expression construct plasmids. Plasmids were preincubated with GeneJuice transfection reagent in OptiMEM medium according to the suggestions of manufacturer. On the next day, cell membranes were stained with FM®4-64 red fluorescent membrane stain. Life cell images were captured in 5% CO2 atmosphere at 37° C. with confocal microscope (Zeiss LSM 710).

Recombinant expression of G434-P548-His6 forms large oligomers in E. coli.

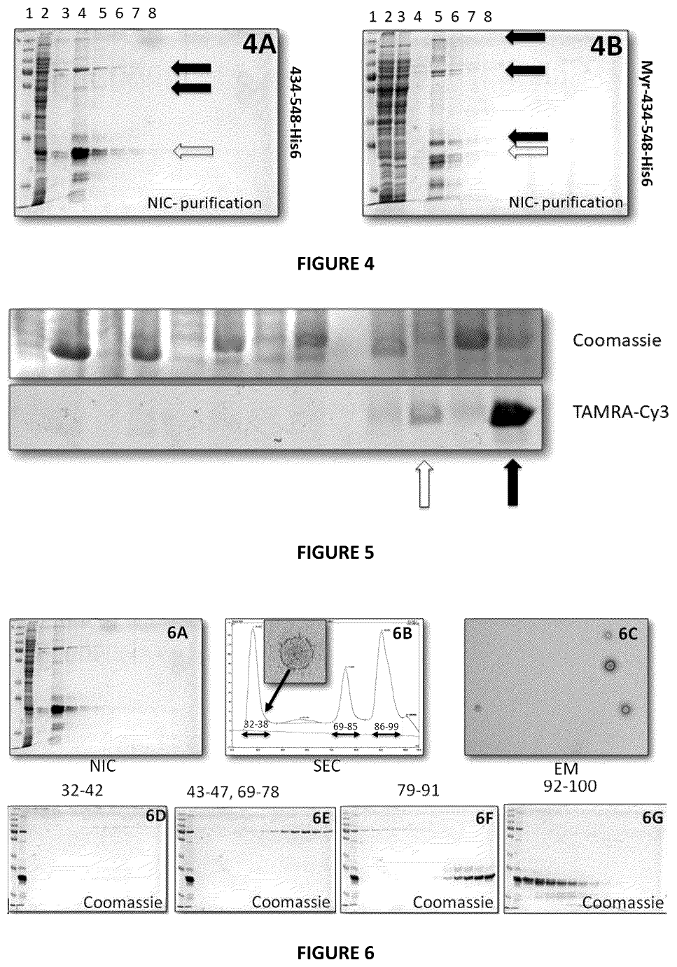

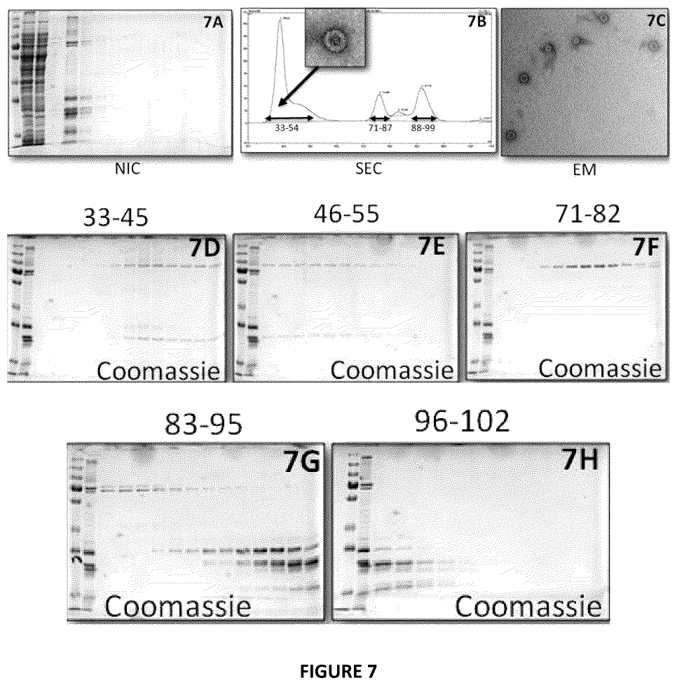

The monomers and oligomers were purified on Nickel affinity column and visualized on Coomassie gels (FIG. 4A). 434-Glycine on the N-terminus (434G) was identified as a substrate for N-myristoyl transferase (NMT). Coexpression of G434-P548-His6 recombinant peptide with CaNMT (Candida albicans N-myristoyl transferase) resulted N-terminal co-translational myristoylation of G434-P548-His6 peptide (FIG. 5). Post-and cotranslational myristoylation of proteins increases protein-protein interaction as well as membrane binding of the modified proteins. Myristoylation forced the oligomerization of Myr-434-548-His6 fragment in E. coli expression system (compare FIG. 4A and 4B). Separation of the unmodified and myristoylated 434-548-His6 oligomers by Size Exclusion Column (SEC) resulted three discrete peaks of the oligomers with different size (FIG. 6B and 7B). However, the Coomassie staining of the SDS-PAGE gels proved proteins only in the fractions of 2^{nd} and 3^{rd} peaks (FIG. 6D-G and 7D-H).

Regarding the retention time of the first peak (10 minutes), it suggested, that largest oligomers might be bigger than 100 000 kDa which fragments could be visualized and detected by electron microscopy using negative staining.

### Co-Expression of CaNMT-pET11b and G434-P548-His6-pET28a in BL21-DE3-pLysS E. coli stream

The BL21 competent E. coli cells were co-transformed with both CaNMT-pET11b and G434-P548-His6-pET28a expression plasmids. The pET11b encodes the Candida Albicans: untagged N-myristoyl-tramferase (CaNMT) protein and Ampicillin resistance gene sequence. However, pET28a plasmid encodes the G434-P548-fragment tagged with C-terminus poly-histidine tag and Kanamycin resistance gene sequence. The double transformed BL21 cells were selected on LB-agar containing the both Ampicillin 100 μg/ml in final and the Kanamycin 50 μg/ml final concentration. For recombinant expression of N-term myristoylated G434-P548-His, E. coli cells were fed with Myristic acid in 500 μM final concentration.

### Purification of Recombinant Protein Tagged with Poly-Histidine Tag

Pellets of 1 Liters of bacterial cultures were frozen on −20° C. Each liter were resuspended in 20 mL of Lysis buffer (300 mM NaCl, 50 mM Na2HPO4xNaH2PO4, 0.25% Triton-X, 0.25% NP40). The lysed samples were homogenized with ultrasound for 3×2 minutes with 50% pulsation, then centrifuged with 14400×g. Meanwhile 1 mL of His trapping Nickel bound agarose beads were washed with 5 cv dH2O and 5 cv Lysis buffer. The supernatant were incubated with the beads overnight on 4° C. shaken gently. The suspension were then applied to gravitational filter columns, washed 25 mL of Lysis buffer and 50 mL Washing buffer (300 mM NaCl, 50 mM Na2HPO4xNaH2PO4), respectively. The proteins then were eluated with 1 mL Elution buffer (300 mM NaCl, 50 mM Na2HPO4xNaH2PO4, 1M Imidazole). The eluates were stored on −20° C.

### SEC Purification of G434-P548-His6 and Myr-G434-P548-His6

Monomers and large oligomers of G434-P548 and Myr-G434-P548 eluted from NIC (HisSelect Nickel affinity column) were separated with Superose™ 6 10/300 GL Size Exclusion Column (SEC) on Hitachi L/7300 HPLC. SEC-fractions were visualized with SDS-PAGE Commassie gel staining.

### Electron Microscopy: TEM

Myristoylated or unmyristoylated truncated BFSP1 Tail (GGQ-548P-His6) were diluted into assembly buffer respectively to 100 μg/ml. A carbon film that had been coated onto freshly cleaved mica was then floated onto the surface of the sample prior to being negatively stained with 1% (w/v) uranyl acetate (Agar Scientific, UK) and retrieved with 400 mesh copper grids (Agar Scientific, UK). Grids were examined in an Hitachi H-7600 transmission electron microscope (Hitachi High-Technologies Corporation, Japan), using an accelerating voltage of 100 kV. Images were acquired using a CCD camera (Advanced microscopy Technology, Danvers, MA) and assembled into montages using Adobe R Photoshop CS (Adobe System. San Jose, CA).

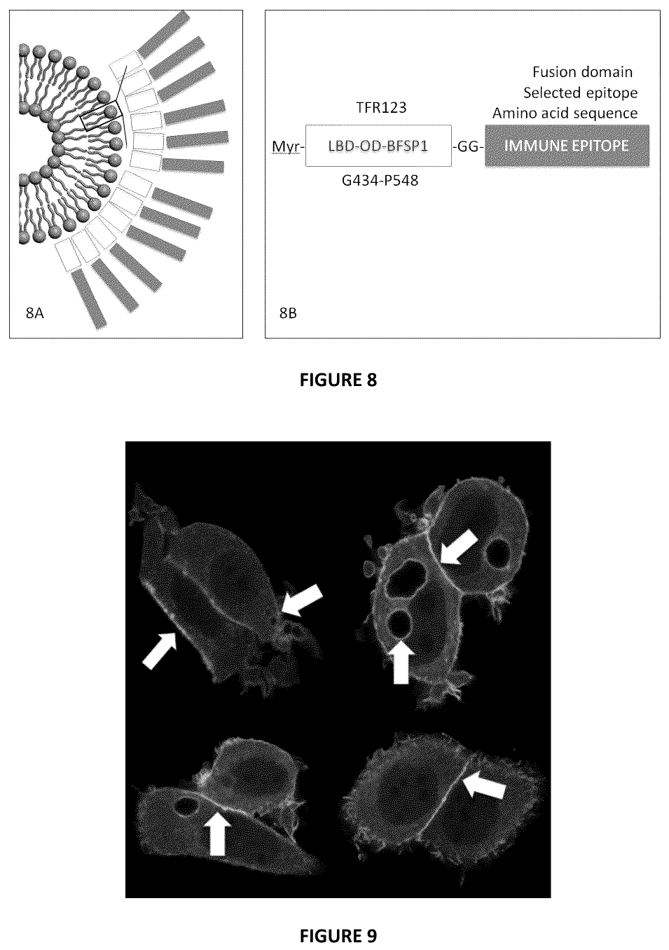

Electron microscopic pictures confirmed the fluorescence microscopic data that the both 434-548-His6 and Myr-434-548-His6 fragments of BFSP1 form very strong oligomers anchored into lipid layers and these oligomers stabilize large membrane vesicles generated by sonication during the harvesting and purification of the recombinant peptide fragment of BFSP1 (FIG. 6B, 6C and FIG. 7B, 7C). The areas under the curve of 2^{nd} and 3^{rd} peaks were decreased due to the myristoylation, meanwhile the area under the curve of the 1^{st} peak was increased due to the co-translational modification (compare FIG. 6B and 7B). Myristoylation established a driving force for both the oligomerization and the membrane binding property of Myr-434-548-His6 fragment in E. coli expression system. The very strong membrane binding capacity and oligomerisation property of human BFSP1-LBD-peptide (G434-P548, or TFR123) increases stability of lipid vesicles extremely. It is resistant to 2% SDS of the Laemmli sample buffer: recombinant G434-P548-His6 peptides anchored into the membrane vesicle could not be released from the membrane even due the detergent (2% SDS), which means there is no remarkable amount of recombinant peptide detected in the Coomassie gel, which peptide represents the collected fractions of the very 1^{st} peak during the SEC purification. This phenomenon could be only due to the very strong recombinant peptide layer on the surface of the artificial lipid vesicles formed by sonication. The G434-P548 peptide monomers (TFR123=LBD-ODBFSP1) can be extended with any kind of immunogen peptide sequence recombinantly (FIG. 8, and for detailed procedure see III.).

The LBD-OD-BFSP1 peptide (Myr-G434-P548=TFR123) provides a novel immunization platform for vaccination, however low immunogenic effect is expected by the liposomes coated with only core peptide (human G434-548-BFSP1 fragment), which might contribute to the safe application of this novel molecular platform for immunization and vaccination techniques (FIG. 8).

### c. LBD (Lipid Binding Domain) G434-E463

The first 30 amino acid on the N-terminus of G434-P548 fragment was a predicted and proved lipid binding domain of BFSP1. The 27 amino acid of LBD (G434-T460) was enough for membrane binding and membrane vesicle formation (FIG. 9). LBD-BFSP1 (G434-E463) is a part of the TFR123 platform. Similar other LBDs of several proteins like PH (Pleckstrin homology domain) superfamily proteins: ARF (ADPribosylation factor), PTEN, PKC, IRS1, Dynamin, OPA, Mitofusin, Pleckstrin etc, may be used in immunization platforms as well. C1-DAG binding superfamily proteins: PKC. AKAP13 etc.; C2-superfamily: PTEN, Synaptotagmin, PLC, PLA etc.

FYVE domain: RhoGEF, EEA1 etc.; PX domain: PLD, PI3K, NOX (NADPH oxidase) etc.; ENTH domain: Epsin, CLINT1 etc.; ANTH domain: HIP1, HIP1R etc.; BAR domain: AMPH, Endophilin etc.; FERM domain: Ezrin, Radixin etc.; PDZ domain: Erbin etc.; Tubby domain: TUB etc.

The LBD of BFSP1 has got many advantages compared to the abovementioned LBD-domain families:
G434-E463 is the shortest LBD-motif
It is modified by myristate co-or post-translationally, inducing more membrane binding and protein-protein interaction (oligomerization)
It hasn't got any enzymatic activity
It is not related to endocytosis
It is not related to vesicle transport
Lipid binding does not require positively charged ions like Calcium or Magnesium
It is not revealed so far as a factor for cell signalling kinases
It is not related to neurotransmission
It is exclusively tissue specific in the avascular eye lens.

G434-E463 is a very unique LBD which seems the most appropriate liposome binding motif and extended with OD of BFSP1 (Oligomerisation domain: L464-P548) it is a membrane-stabilizing polymer without possible side effects due to tissue specificity and the original function of BFSP1, which is preserving the membrane structure of fibre cells and transparency of the eye lens.

### d. OD (Oligomerisation Domain) L464-P548 of BFSP1

Protein folding is a critical point to gain the appropriate structure and function of proteins. Polymerization of the cytoskeletal elements is a tightly regulated process involving several regulatory factors like interacting proteins, PTM (post-translational modifications) etc. Recombinant intermediate filament proteins (like BFSP1, BFSP2 or GFAP) are assembled “in vitro” buffers very well (Exp Cell Res. 2007 Jun. 10: 313(10): 2180-2188.doi:10.1016/j.yexcr.2007.04.005.). The recombinant full-length BFSP1 (M1-S665) goes into the inclusion body, and this protein can be re-solubilized with 8M urea. The stepwise removal of the unfolding reagent (Urea) induces self assembly of BFSP1 filaments. Recombinant OD-BFSP1 (L464-P548) as well as the G434-P548: TFR123 do not go into the inclusion body, those recombinant peptides remain soluble in E. coli cells. The soluble peptide fragments provide an easier and cheaper purification protocol for recombinant LBD-OD-BFSP1: G434-P548: TFR123.

The C-terminal truncation of OD could decrease the oligomerization property, which allows us to adjust the strength of the oligomer peptide layer anchored into the liposomes.

### II. Liposomes

### a. Lipid Composition

The complex lipid components of the artificial liposomes mimic the native lipid bilayer membranes.

18:1 (DELTA 9-CIS) PC (DOPC)

N-Palmitoyl-D-Sphingomyelin

1,2-Dioleyl-SN-Glycero-3-Phosphoethanol

Cholesterol

### b. Producing Uniform Liposomes with Sonication and Extruder

The purified lipids were stored in chloroform:methanol (2:1) solvent. The suspension was distilled under inert gas, then in order to remove the excess solvent, the remaining content was dried under vacuum as long as one hour. The lipids were then hydrated in the desired aqueous buffer above melting temperature, afterwards we acquired multilamellar vesicles (MLV) following a series of a repetitive freezing-thawing in liquid Nitrogen. The MLV suspension then were sonicated until transparency, acquiring small unilamellar vesicles (SUV, d˜20-40 nm). The MLV system was filtered with an extrusion procedure (LiposoFast, Avestin, Inc.) using a polycarbonate filter (pore diameter: 100 and 200 nm). Filtered multiple times we achieved well established sized, round, unilamellar vesicles (LUV).

### III. Cloning the TFR123-SARS-CoV-2 Recombinant Peptide Expression System

### a. TFR123-pET28a: Core Peptide Expression System (G434-P548-His-pET28a)

Nucleotide sequence of G434-P548=LBD-OD-BFSP1 was amplified by PCR using custom DNA oligonucleotide (Table 2.)

### Table 2

| Name of PCR primer | Nucleotide sequence 5′-3′ |
|---|---|
| N′-NcoI-G434-FOR | CCATGGGAGGGCAGATAAGCAAAGGC |
| (SEQ ID NO: 52) | |
| 3′-XhoI-P548-REV | CTCGAGTGGCTCAATCTCCTTGTCTA |
| (SEQ ID NO: 53) | TAGG |

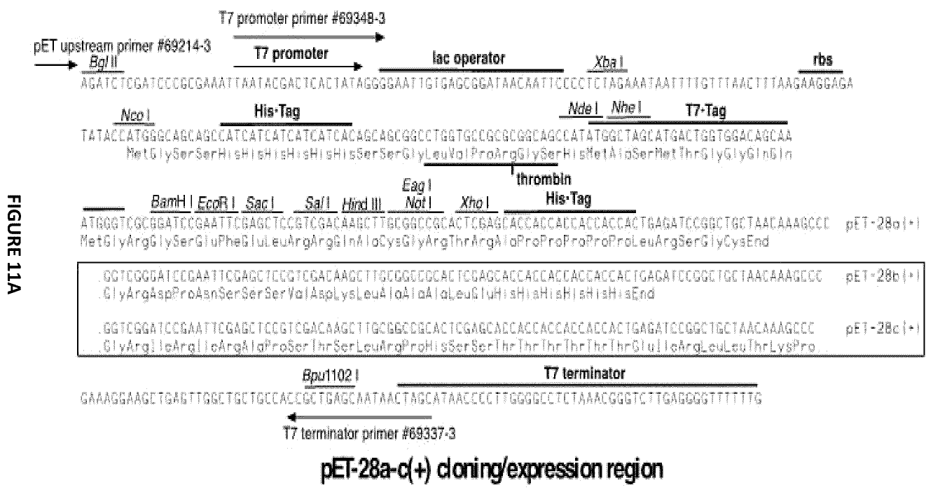

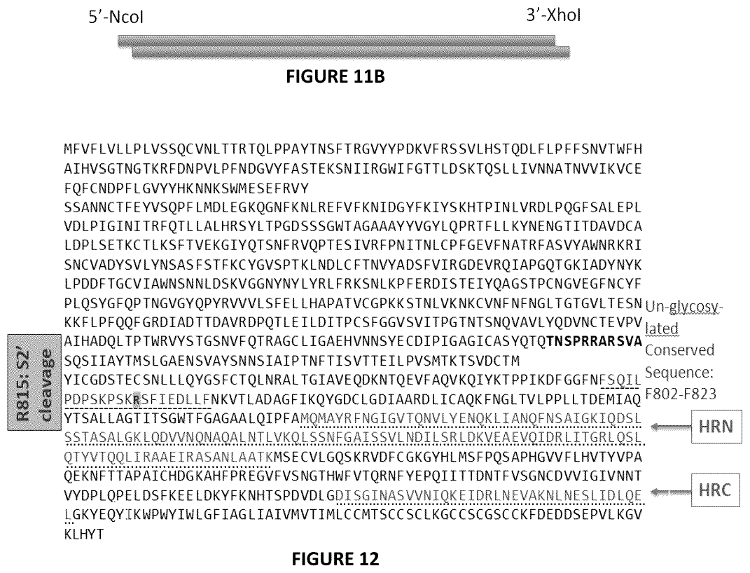

The forward primer introduced a 5′ NcoI restriction endonuclease site, however the reverse primer excludes the endogenous “stop” codon and introduces a 3′ XhoI site on the PCR amplicon. Image clone of full-length BFSP1-pET23a was used for template DNA during the PCR reaction. PCR amplicon was inserted into pGEMT-easy cloning vector (Promega). The correct nucleotide sequence of amplicon was confirmed by DNA sequencing using T7 and Sp6 primers. Confirmed TFR123-pGEMTeasy clone was digested with NcoI-XhoI enzymes. The 345 bp DNA insert was run on 1.5% acryl amide gel and purified with QIAquick gel extraction kit from Qiagen. Destination vector (pET28a) was double digested with the same NcoI-XhoI restriction endonucleases. The 5′-NcoI-G434-P548-XhoI-3′ DNA insert was ligated into pET28a vector digested with NcoI-XhoI (FIG. 11). Restriction enzymes and T4-DNA Ligase were from Neb (New England Biolab). The TFR123-pET28a were transformed into DH5-Alpha and BL21-DE3-pLysS competent E. coli stream.

### b. Best Candidate Epitope Sequences for TFR123-SARS-CoV-2

The most significant challenge of SARS-CoV-2 vaccine is to identify the most useful immunogen epitope sequence of the virus. Spike protein of SARS-CoV-2 seems to be the most obvious antigen. There are many strategies and challenges to use Spike protein for vaccination.

There are three remarkable problems regarding Spike protein.

Nr.1: Spike is a highly glycosylated protein (see Watanabe et al., Science 369, 330-333 (2020) 17 July). Post translational modification excludes the cost effective and high yield recombinant expression of Spike protein in bacteria. However, glycosylation might be variable changing slightly the 3D structure of the Spike. This decreases the reproducible immunogen effect of the possible vaccine. Eukaryotic expression (for instance in HEK293 cells) of Spike protein is expensive and the problem with variable glycosylation is still there.

Nr.2: The outer segment of Spike: S1-protein, which comprises the receptor binding domain (RBD) too, does not have as conserved an amino acid sequence as the S2-protein and the transmembrane domain (TM). It means that evolutional variation of S1-protein might decrease the reproducible immunogen effect of the possible vaccine.

Nr.3: Unmodified (no glycosylation) conserved amino acid sequences are located in hidden regions of Spike, which is useless for vaccination, because those amino acid structures are not well displayed on the Spike's surface.

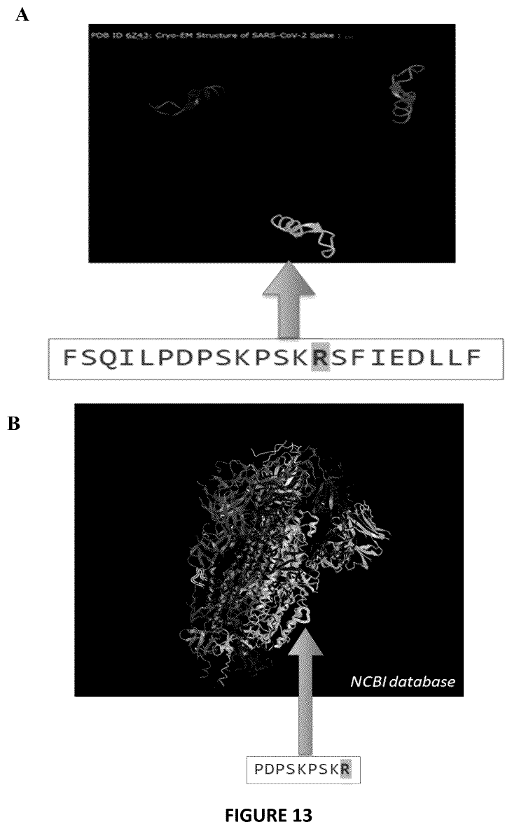

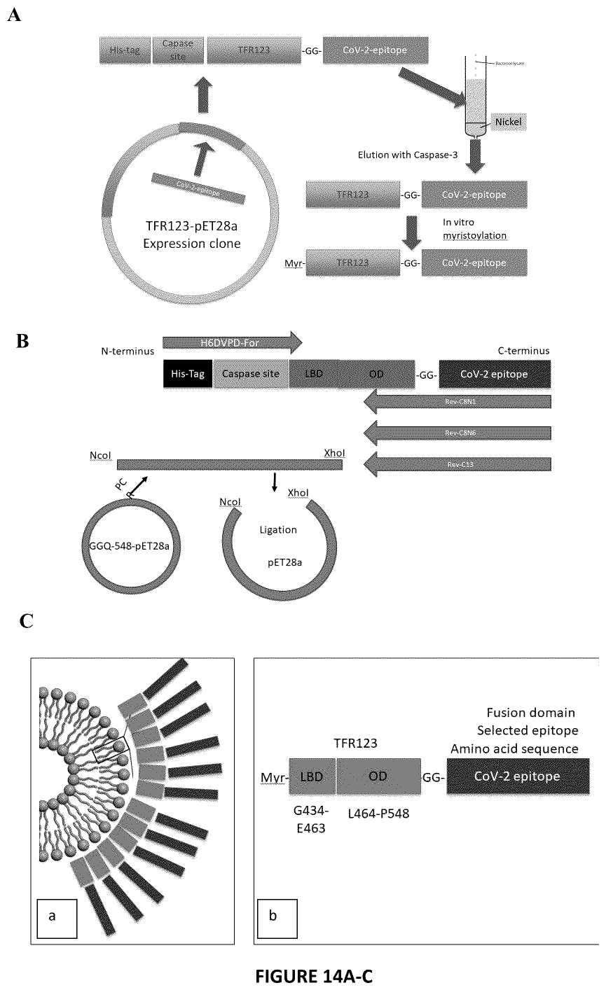

Investigation of the amino acid sequence of Spike protein suggest a very promising sequence: F802-F823 (PBM: protease binding motif) which comprises the S2′ proteolytic cleavage point (FIG. 13). F802-F823 amino acid sequence has no Asparagine (“N”) which excludes the N-glycosylation. This is very important because N-glycosylation of spike protein is performed on Asparagine residues (Watanabe et al., Science 369, 330-333 17 Jul. 2020). Each monomer of trimeric Spike displays 22 N-glycosylation sites (see Watanabe et al., Science 369, 330-333 17 Jul. 2020). Glycosylation seems essential for internalization of virus into the pneumocytes, because disruption of Spike glycosylation impairs viral entry (Genetic Engineering and Biotechnology News. Retrieved 2020 May 18). Ligand binding function of ACE2 receptors of pneumocytes is very similar to lectin receptors recognising carbohydrate modification of proteins indicating an importance of glycan-protein interactions in the viral entry. However, substrate-binding of proteases prefer unmodified amino acid sequences. Proteases need clear access to the cleavage site of the substrate. In fact, glycosylation could mask this access. On the other hand, protease binding site of substrate must be very well displayed, otherwise proteolytic cleavage will not happen. The S2′ cleavage site of Spike protein might be an excellent suitable epitope to raise SARS-CoV-2 vaccine, because it is very well displayed on the outer segment of Spike protein furthermore it is no glycosylated (FIG. 13).

TFR123 immunization platform is capable to display extremely short peptide epitopes in very high concentration. Liposomes with a diameter of 200 nanometres and coated by Myr-G434-P548-SARS-CoV-2-S2′-cleavage site might be very stable and immunogen. TFR123-SARS-CoV-2-S2′-cleavage site could become to be a very promising new COVID19 vaccine.

The optimal size of the immunogen epitope sequence contains 7-14 amino acids, which is small enough to have no influence in oligomerization and lipid binding of TFR123 immunization platform. For that reason, an entire clone family was designed (see below: III.b.).

TFR 123-fusion peptide is sub-cloned into pET28a expression vector. Poly-histidine tag and Caspase-3 cleavage site is designed on the N-terminus of the TFR123-fusion peptide, because it is to be purified by Nickel affinity column (His-Select affinity gel from Sigma-Aldrich) and the untagged recombinant fusion peptide is to be eluted by recombinant activated Caspase-3 (ThermoFisher) from the Nickel-column (NIC). In vitro myristoylation of the fusion peptide is achieved by Candida albicans recombinant N-myristoyl-transferase (CaNMT) and activated myristate (Myr-S-CoA). Myristoylated fusion peptide is separated by HPLC-SEC (Size Exclusion Column).

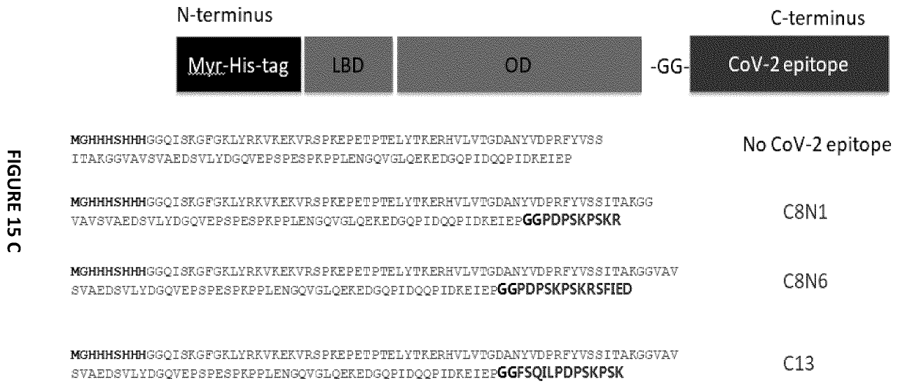

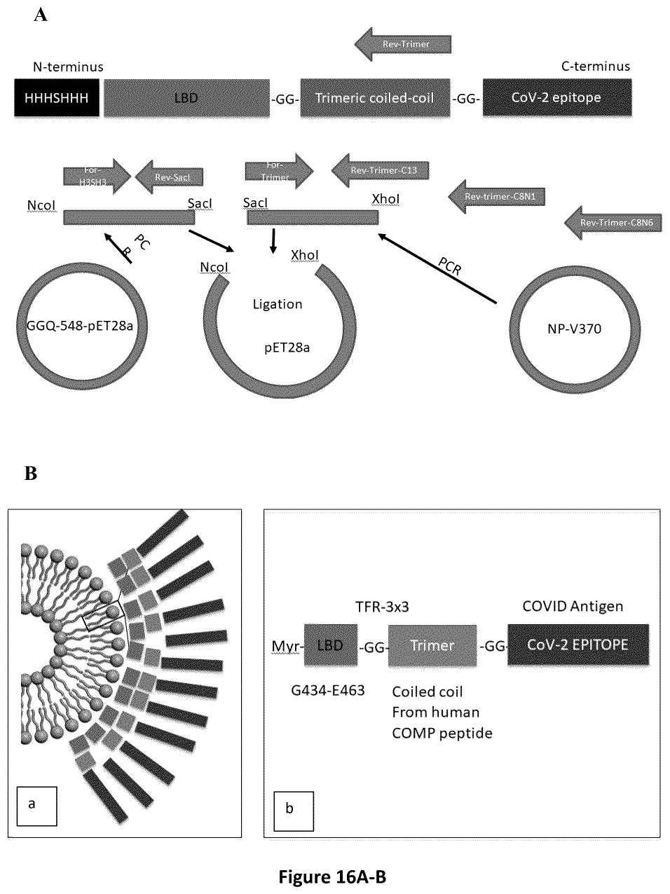

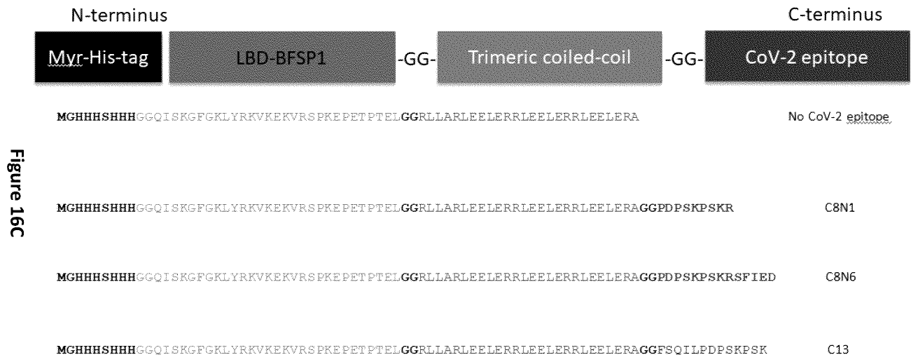

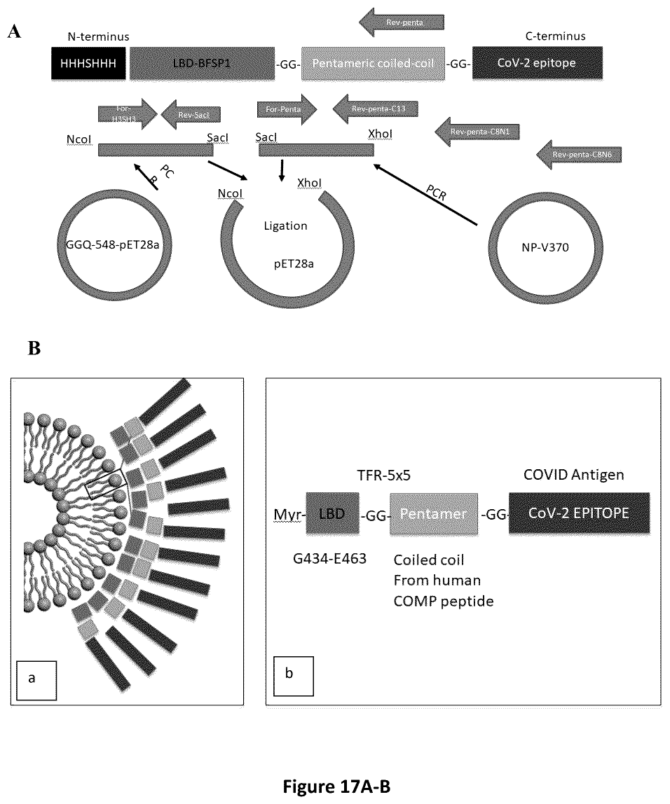

Cloning strategy of TFR123-fusion peptide: (FIG. 16).

### i. TFR123 the Core

The already existing G434-P548-His6-pET28a expression clone has to be modified by N-terminal Histidine tag and Caspase cleavage site recombinantly. These can be performed by PCR or by predesigned oligonucleotides hybridized and fused to the original G434-P548 coding gene sequence. We achieved both cloning strategies. For PCR primer sequences see table 3. For the schematic structure of the PCR amplicon see FIG. 19.

Table 3. PCR primers for TFR123 core construct: Forward primer introduces a NcoI site and poly-Histidine tag coding sequence onto the 5′-end of the G434-P548 coding sequence. Reverse primer introduces a “TAA” stop codon and a XhoI site onto the 3′-end of the original core peptide coding sequence. The meaning of this cloning is to remove C-term His-tag and to introduce a N-term His-tag and caspase cleavage site.

| Name of PCR primer | Sequence 5′-3′ |
|---|---|
| H3SH3-FOR | ccATGGGCcaccaccacagccaccac |
| (SEQ ID NO: 30): | cacggagggcagataagcaaaggc |
| NOHis REV | CTCGAGTTAtggctcaatctccttgt |
| (SEQ ID NO: 31): | ctatagg |

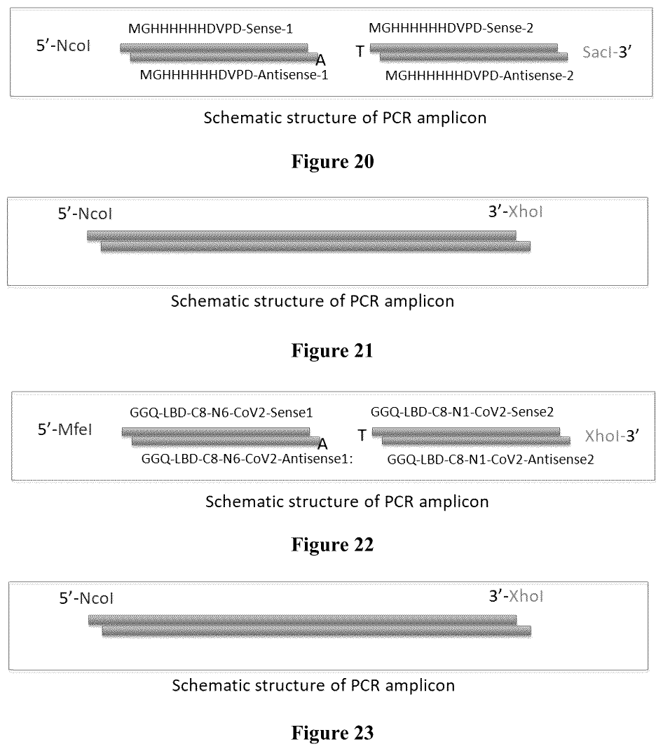

For sequences of Hybridization strategy see table 4. For the schematic structure of the PCR amplicon see FIG. 20.

Table 4. Nucleotide sequences for TFR123 core (His6-Casp3-G434-P548-pET28a): The table contains the nucleotide sequences hybridized to each other (Sense-1 to Antisense1, or Sense-2 to antisense-2). Hybridized double stranded DNAs having compatible ends to each other as well as to the destination vector G434-P548-pET28a digested with NcoI-SacI.

| Name of oligo | Nucleotide sequence 5′ to 3′ |
|---|---|
| MGHHHHHHDVPD- | CATGGGCcaccaccaccaccaccacgatg |
| Sense-1 | tgccagatggagggcagataagcaaaggc |
| (SEQ ID NO: 54): | tttgggaaacta |
| MGHHHHHHDVPD- | agtttcccaaagcctttgcttatctgccc |
| Antisense-1 | tccatctggcacatcgtggtggtggtggt |
| (SEQ ID NO: 55): | ggtggCC |
| MGHHHHHHDVPD- | tacaggaaggtcaaggagaaagtgagaag |
| Sense-2 | ccccaaagagcctgagacccccactgagc |
| (SEQ ID NO: 56): | t |
| MGHHHHHHDVPD- | Cagtgggggtctcaggctctttggggctt |
| Antisense-2 | ctcactttctccttgaccttcctgtat |
| (SEQ ID NO: 57): | |

### ii. TFR123-C8-N1 (TFR123-P807-R815)

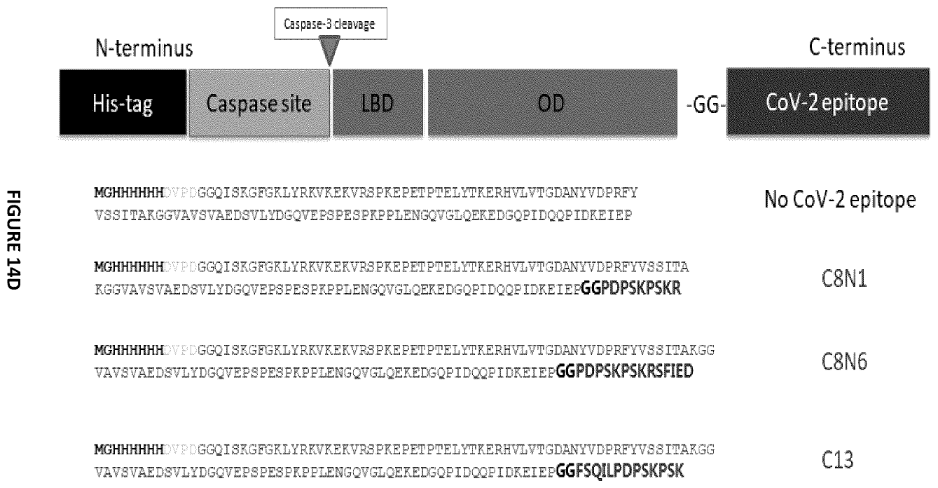

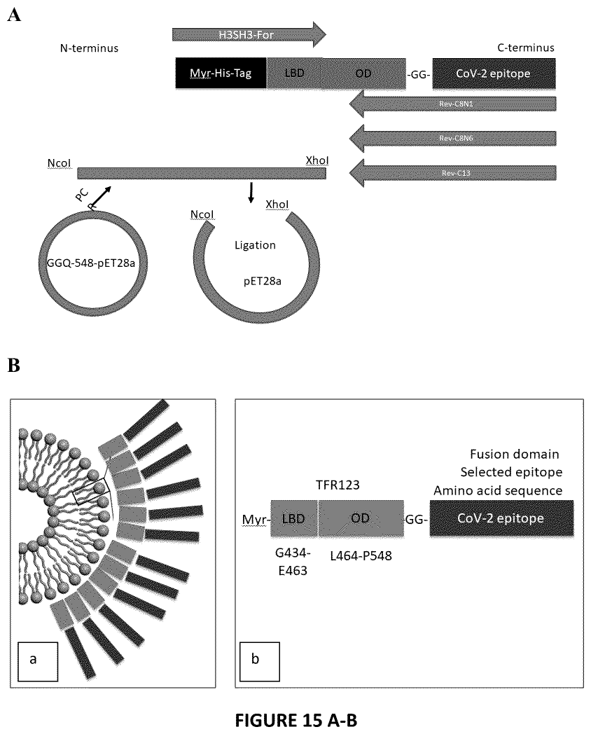

The best candidate sequence is the outer loop (P807-R815) of the S-protein (FIG. 15C, Table 19). To design TFR123-P807-R815 clone, the already existing TFR123 core (designed in: III.b.i) has to be modified by C-terminal extension with P807-R815 amino acid coding sequence of SARS-CoV-2 S-protein. These can be performed by PCR or by predesigned oligonucleotides hybridized and fused to the TFR123 core coding gene sequence designed in III.b.i. We achieved both cloning strategies. For PCR-primer sequences see table 5. For the schematic structure of the PCR amplicon see FIG. 21.

Table 5. PCR primers for TFR123-P807-R815 construct (C8-N1): Forward primer introduces a NcoI site and poly-Histidine tag coding sequence onto the 5′-end of the G434-P548 coding sequence. Reverse primer introduces the outer loop of S2′ cleavage site of S-protein (P807-R815) a “TAA” stop codon and a XhoI site onto the 3′-end of the original core peptide coding sequence.

| Name of PCR primer | Sequences 5′ to 3′ |
|---|---|
| H6DVPD-FOR | CCATGGGCcaccaccaccaccaccacgatg |
| (SEQ ID NO: 46): | tgccagatggagggcagataagcaaaggc |
| C8-N1-REV | ctcgagTTAcctcttgcttggttttgatgg |
| (SEQ ID NO: 32): | atctggCCCTCCtggctcaatctccttgtc |
| | tatag |

For sequences of Hybridization strategy see table 6. (Note: The primers called “GGQ-LBD-C8-N6-CoV2-Sense1” and “GGQ-LBD-C8-N6-CoV2-Antisense1” are used for the both TFR123-P807-D820 and TFR123-P807-R815 constructs.) For the schematic structure of the PCR amplicon see FIG. 22.

Table 6. Nucleotide sequences for TFR123-P807-R815-pET28a construct (C8-N1): The table contains the nucleotide sequences hybridized to each other (Sense-1 to Antisense1, or Sense-2 to antisense-2). Hybridized double stranded DNAs having compatible ends to each other (-T and -A overhangs) as well as to the destination vector TFR 123-pET28a digested with MfeIXhoI.

| Name of oligo | Nucleotide sequence 5′ to 3′ |
|---|---|
| GGQ-LBD-C8-N6-CoV2- | aattgaccagcagcctatagacaaggag |
| Sense1 (SEQ ID | attgagccaGGAGGGcca |
| NO: 58): | |
| GGQ-LBD-C8-N6-CoV2- | ggCCCTCCtggctcaatctccttgtcta |
| Antisense1 (SEQ ID | taggctgctggtc |
| NO: 59): | |
| GGQ-LBD-C8-N1-CoV2- | gatccatcaaaaccaagcaagaggTAAc |
| Sense2 (SEQ ID | |
| NO: 60): | |
| GGQ-LBD-C8-N1-CoV2- | tcgagTTAcctcttgcttggttttgatg |
| Antisense2 (SEQ ID | gatct |
| NO: 61): | |

### iii. TFR123-C8-N6 (TFR123-P807-D820)

The second-best epitope sequence is the C-terminal extended version of the outer loop of S-protein. To design and create TFR123-P807-D820 clone, same procedure was achieved as in the case of the previous clone: TFR 123-P807-R815. For PCR-primer sequences see table 7. For the schematic structure of the PCR amplicon see FIG. 23.

Table 7. PCR primers for TFR123-P807-D820 construct (C8-N6): Forward primer introduces a NcoI site and poly-Histidine tag coding sequence onto the 5′-end of the G434-P548 coding sequence. Reverse primer introduces the C-terminal extension of the outer loop of S2′ cleavage site of S-protein (P807-D820) a “TAA” stop codon and a XhoI site onto the 3′-end of the original core peptide coding sequence.

| Name of PCR | |
|---|---|
| primer | Sequences 5′ to 3′ |
| H6DVPD-FOR | CCATGGGCcaccaccaccaccaccacgatg |
| (SEQ ID NO: 46): | tgccagatggagggcagataagcaaaggc |
| C8-N6-REV | ctcgagTTAatcttcaataaatgacctctt |
| (SEQ ID NO: 33): | gcttggttttgatggatctggCCCTCCtgg |
| | ctcaatctccttgtctatag |

For sequences of Hybridization strategy see table 8.

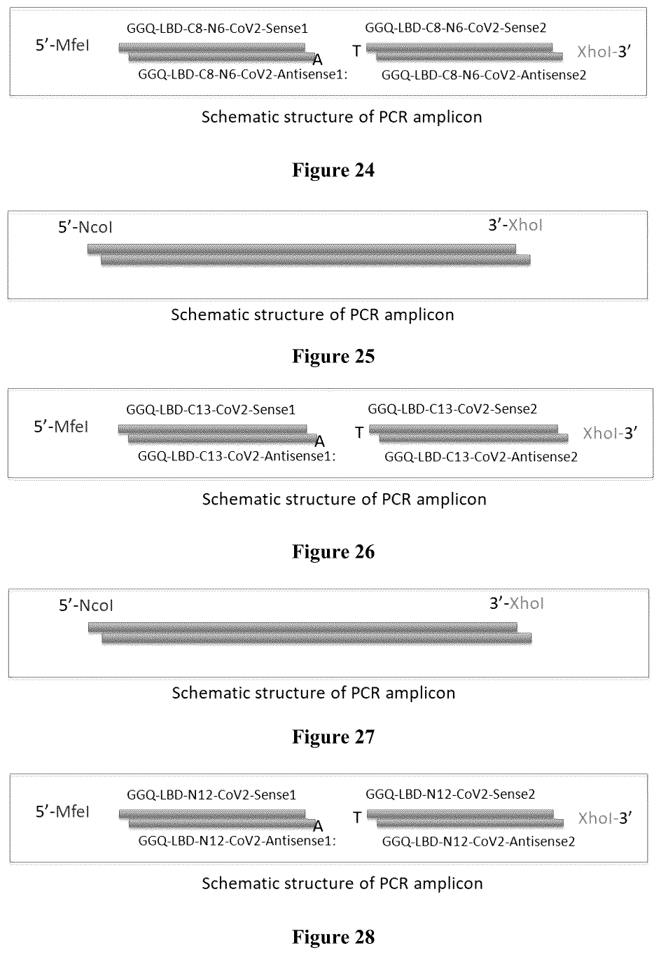

(Note for table 8: The primers called “GGQ-LBD-C8-N6-CoV2-Sense1” and “GGQLBD-C8-N6-CoV2-Antisense1” are used for the both TFR123-P807-D820 and TFR123-P807-R815 constructs.) For the schematic structure of the PCR amplicon see FIG. 24.

Table 8. Nucleotide sequences for TFR123-P807-R820-pET28a construct (C8-N6): The table contains the nucleotide sequences hybridized to each other (Sense-1 to Antisense1, or Sense-2 to antisense-2). Hybridized double stranded DNAs having compatible ends to each other (-T and -A overhangs) as well as to the destination vector TFR 123-pET28a digested with MfeIXhoI.

| Name of oligo | Nucleotide sequence 5′ to 3′ |
|---|---|
| GGQ-LBD-C8-N6-CoV2- | aattgaccagcagcctatagacaaggag |
| Sense1 (SEQ ID | attgagccaGGAGGGcca |
| NO: 58): | |
| GGQ-LBD-C8-N6-CoV2- | ggCCCTCCtggctcaatctccttgtcta |
| Antisense1 (SEQ ID | taggctgctggtc |
| NO: 59): | |
| GGQ-LBD-C8-N6-CoV2- | gatccatcaaaaccaagcaagaggtcat |
| Sense2 (SEQ ID | ttattgaagatTAAc |
| NO: 64): | |
| GGQ-LBD-C8-N6-CoV2- | tcgagTTAatcttcaataaatgacctct |
| Antisense2 (SEQ ID | tgcttggttttgatggatct |
| NO: 65): | |

### iv. TFR123-C13 (TFR123-F802-R815)

The third-best epitope candidate is the N-terminal extension of the outer loop of S-protein. For design and creation of TFR123F-802-R815 clone, the same protocol was performed as previously described in III.b.ii. For PCR-primer sequences see table 9. For the schematic structure of the PCR amplicon see FIG. 25.

Table 9. PCR primers for TFR123-P802-R815 construct (C13): Forward primer introduces a NcoI site and poly-Histidine tag coding sequence onto the 5′-end of the G434-P548 coding sequence. Reverse primer introduces the N-terminal extension of outer loop of S2′ cleavage site of S-protein (P802-R815) a “TAA” stop codon and a XhoI site onto the 3′-end of the original core peptide coding sequence.

| Name of PCR | |
|---|---|
| primer | Sequences 5′ to 3′ |
| H6DVPD-FOR (SEQ | CCATGGGCcaccaccaccaccaccacgatgtg |
| ID NO: 46): | ccagatggagggcagataagcaaaggc |
| C13-REV (SEQ ID | ctcgagTTActtgcttggttttgatggatctg |
| NO: 34): | gtaatatttgtgaaaaCCCTCCtggctcaatc |
| | tccttgtctatag |

For sequences of Hybridization strategy see table 10. For the schematic structure of the PCR amplicon see FIG. 26.

Table 10. Nucleotide sequences for TFR123-P802-R815-pET28a construct (C13): The table contains the nucleotide sequences hybridized to each other (Sense-1 to Antisense1, or Sense-2 to antisense-2). Hybridized double stranded DNAs having compatible ends to each other (-T and -A overhangs) as well as to the destination vector TFR123-pET28a digested with MfeIXhoI.

| Name of oligo | Nucleotide sequence 5′ to 3′ |
|---|---|
| GGQ-LBD-C13-CoV2-Sense1 | aattgaccagcagcctatagacaaggagattgagc |
| (SEQ ID NO: 66): | caGGAGGGttttca |
| GGQ-LBD-C13-CoV2-Antisense1 | gaaaaCCCTCCtggctcaatctccttgtctatagg |
| (SEQ ID NO: 67): | ctgctggtc |
| GGQ-LBD-C13-CoV2-Sense2 | caaatattaccagatccatcaaaaccaagcaagag |
| (SEQ ID NO: 68): | gTAAc |
| GGQ-LBD-C13-CoV2-Antisense2 | tcgagTTAcctcttgcttggttttgatggatctgg |
| (SEQ ID NO: 69): | taatatttgt |

Also, we found that F802-K814 might be a good epitope. C13 therefore may refer to both F802-R815 and F802-K814. F802-K814 is the preferred sequence (SEQ ID NO: 11).

### c. Control Epitope Sequences to Confirm the Best Candidate Epitopes of SARSCoV-2

i. TFR123-FP (Fusion peptide: S816-F823: SFIEDLLF) extended up to N12. Fusion Peptide (FD) comprises only 8 amino acids (S816-F823). The 3D structure analysis of SARS-CoV-2 S-protein revealed that this sequence is located in a hidden region in the closed position of Spike protein. Although FP will be well displayed after S2′ cleavage (open position of Spike) it seems to be not the best epitope sequence for vaccination. Despite the positional disadvantages of FP, it is capable to prove our hypothesis, why the outer loop of S2′ cleavage site might be the best epitope candidate for vaccination. For immunological investigations, extended FP sequence was designed for TFR123 platform (TFR123-R815-V826). For cloning, the same procedure was achieved as in the case of the previous TFR123 clones. For PCR-primer sequences see table 11. For the schematic structure of the PCR amplicon see FIG. 27.

Table 11. PCR primers for TFR123-R815-V826 construct (N12): Forward primer introduces a NcoI site and poly-Histidine tag coding sequence onto the 5′-end of the G434-P548 coding sequence. Reverse primer introduces the N-terminal extension of outer loop of S2′ cleavage site of S-protein (R815-V826) a “TAA” stop codon and a XhoI site onto the 3′-end of the original core peptide coding sequence.

| Name of PCR | |
|---|---|
| primer | Sequences 5′ to 3′ |
| H6DVPD-FOR(SEQ | CCATGGGCcaccaccaccaccaccacgatgt |
| ID NO: 46): | gccagatggagggcagataagcaaaggc |
| N12-REV (SEQ ID | ctcgagTTAcactttgttgaaaagtagatct |
| NO: 48): | tcaataaatgacctCCCTCCtggctcaatct |
| | ccttgtctataggctgctg |

For sequences of Hybridization strategy see table 12. For the schematic structure of the PCR amplicon see FIG. 28.

Table 12. Nucleotide sequences for TFR123-R815-V826-pET28a construct (N12): The table contains the nucleotide sequences hybridized to each other (Sense-1 to Antisense1, or Sense-2 to antisense-2). Hybridized double stranded DNAs having compatible ends to each other (-T and -A overhangs) as well as to the destination vector TFR 123-pET28a digested with MfeIXhoI.

| Name of oligo | Nucleotide sequence 5′ to 3′ |
|---|---|
| GGQ-LBD-N12-CoV2-Sense1 | aattgaccagcagcctatagacaaggagattgagccaGGAGGGa |
| (SEQ ID NO: 70): | |
| GGQ-LBD-N12-CoV2-Antisense1 | CCCTCCtggctcaatctccttgtctataggctgctggtc |
| (SEQ ID NO: 72): | |
| GGQ-LBD-N12-CoV2-Sense2 | ggtcatttattgaagatctacttttcaacaaagtgTAAc |
| (SEQ ID NO: 72): | |
| GGQ-LBD-N12-CoV2-Antisense2 | tcgagTTAcactttgttgaaaagtagatcttcaataaatgacct |
| (SEQ ID NO: 73): | |

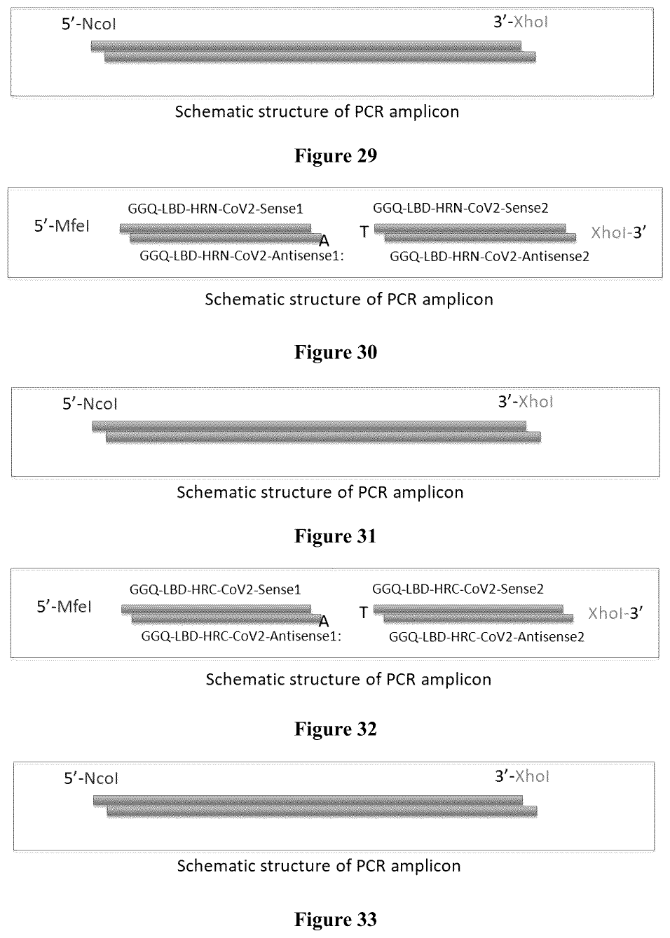

ii. TFR123-HRN (N-terminal heptad repeat: HR1) K921-1934 The HRN (alternative name: HR1) has a very stable coiled-coil structure. It seems an ideal epitope at first sight. Moreover, closed glycosylation sites are faraway (N801 and N1074). However, coiledcoil structure assembles very tightly masking each other. In fact, HR1 is not an optimal epitope, but it can become a suitable control sequence. For cloning, the same procedure was achieved as in the case of the previous TFR123 clones. For PCR-primer sequences see table 13. For the schematic structure of the PCR amplicon see FIG. 29.

Table 13. PCR primers for TFR123-K921-1934 construct (HR1): Forward primer introduces a NcoI site and poly-Histidine tag coding sequence onto the 5′-end of the G434-P548 coding sequence. Reverse primer introduces the N-terminal extension of outer loop of S2′ cleavage site of S-protein (K921-1934) a “TAA” stop codon and a XhoI site onto the 3′-end of the original core peptide coding sequence.

| Name of PCR | |
|---|---|
| primer | Sequences 5′ to 3′ |
| H6DVPD-FOR | CCATGGGCcaccaccaccaccaccacgatgtgcca |
| (SEQ ID NO: | gatggagggcagataagcaaaggc |
| 46): | |
| HRN-REV (SEQ | ctcgagTTAaattttgccaatagcactattaaatt |
| ID NO: 49): | ggttggcaatcaatttCCCTCCtggctcaatctcc |
| | ttgtctataggctgctg |

For sequences of Hybridization strategy see table 14. For the schematic structure of the PCR amplicon see FIG. 30.

Table 14. Nucleotide sequences for TFR123-K921-1934-pET28a construct (HR1): The table contains the nucleotide sequences hybridized to each other (Sense-1 to Antisense1, or Sense-2 to antisense-2). Hybridized double stranded DNAs having compatible ends to each other (-T and -A overhangs) as well as to the destination vector TFR123-pET28a digested with MfeIXhoI.

| | Nucleotide sequence |
|---|---|
| Name of oligo | 5′ to 3′ |
| GGQ-LBD-HRN-CoV2-Sense1 | aattgaccagcagcctatagac |
| (SEQ ID NO: 74): | aaggagattgagccaGGAGGGc |
| | ca |
| GGQ-LBD-HRN-CoV2-Anti- | ggCCCTCCtggctcaatctcct |
| sense1 (SEQ ID NO: 75): | tgtctataggctgctggtc |
| GGQ-LBD-HRN-CoV2-Sense2 | gatccatcaaaaccaagcaaga |
| (SEQ ID NO: 76): | ggTAAc |
| GGQ-LBD-HRN-CoV2-Anti- | tcgagTTAcctcttgcttggtt |
| sense2 (SEQ ID NO: 77): | ttgatggatct |

iii. TFR123-HRC (C-terminal heptad repeat: HR2) A1174-N1187 The HRC (alternatively: HR2) is a suboptimal sequence for vaccination, because it is flanked with glycosylation sites (N1173 and N1194) and it is located very closed to the TM (Transmembrane Motif). For cloning, the same procedure was achieved as in the case of the previous TFR123 clones. For PCR-primer sequences see table 15. For the schematic structure of the PCR amplicon see FIG. 31.

Table 15. PCR primers for TFR123-A1174-N1187 construct (HR2): Forward primer introduces a NcoI site and poly-Histidine tag coding sequence onto the 5′-end of the G434-P548 coding sequence. Reverse primer introduces the N-terminal extension of outer loop of S2′ cleavage site of S-protein (A1174-N1187) a “TAA” stop codon and a XhoI site onto the 3′-end of the original core peptide coding sequence.

| Name of PCR | |
|---|---|
| primer | Sequences 5′ to 3′ |
| H6DVPD- | CCATGGGCcaccaccaccaccaccacgatgtgcca |
| FOR (SEQ ID | gatggagggcagataagcaaaggc |
| NO: 46): | |
| HRC-REV | ctcgagTTAattgaggcggtcaatttctttttgaa |
| (SEQ ID NO: | tgtttacaactgaagcattCCCTCCtggctcaatc |
| 50): | tccttgtctataggctgctg |

For sequences of Hybridization strategy see table 16. For the schematic structure of the PCR amplicon see FIG. 32.

Table 16. Nucleotide sequences for TFR123-A1174-N1187-pET28a construct (HR2): The table contains the nucleotide sequences hybridized to each other (Sense-1 to Antisense1, or Sense-2 to antisense-2). Hybridized double stranded DNAs having compatible ends to each other (-T and -A overhangs) as well as to the destination vector TFR123-pET28a digested with MfeIXhoI.

| | Nucleotide sequence |
|---|---|
| Name of oligo | 5′ to 3′ |
| GGQ-LBD-HRC-CoV2-Sense1 | aattgaccagcagcctatag |
| (SEQ ID NO: 78): | acaaggagattgagccaGGA |
| | GGGaatgct |
| GGQ-LBD-HRC-CoV2-Antisense1 | gcattCCCTCCtggctcaat |
| (SEQ ID NO: 79): | ctccttgtctataggctgct |
| | ggtc |
| GGQ-LBD-HRC-CoV2-Sense2 | tcagttgtaaacattcaaaa |
| (SEQ ID NO: 80): | agaaattgaccgcctcaatT |
| | AAc |
| GGQ-LBD-HRC-CoV2-Antisense2 | tcgagTTAattgaggcggtc |
| (SEQ ID NO: 81): | aatttctttttgaatgttta |
| | caactgaa |

iv. TFR123-RBM (Receptor Binding Motif: A475-C488) The RBM is the ligand-interaction part of the RBD (Receptor binding domain: N331-P527) of S-protein. It seems a very promising epitope, because neutralizing antibodies raised by immunization of RBM-antigen could prevent the docking and entry of the virus. On the other hand, the position of RBM in the structure of S-protein is located in the groove of the RBD (Receptor Binding Domain). It means RBM is not very well displayed sequence of Spike protein. For that reason, RBM will be used as a control sequence in order to prove our best epitope candidates. For cloning, the same procedure was achieved as in the case of the previous TFR123 clones. For PCR-primer sequences see table 17. For the schematic structure of the PCR amplicon see FIG. 33.

Table 17. PCR primers for TFR123-A475-C488 construct (RBM): Forward primer introduces a NcoI site and poly-Histidine tag coding sequence onto the 5′-end of the G434-P548 coding sequence. Reverse primer introduces the N-terminal extension of outer loop of S2′ cleavage site of S-protein (A475-C488) a “TAA” stop codon and a XhoI site onto the 3′-end of the original core peptide coding sequence.

| Name of PCR | |
|---|---|
| primer | Sequences 5′ to 3′ |
| H6DVPD- | CCATGGGCcaccaccaccaccaccacgatgtgcca |
| FOR (SEQ ID | gatggagggcagataagcaaaggc |
| NO: 46): | |
| RBM-REV | ctcgagTTAacaattaaaaccttcaacaccattac |
| (SEQ ID NO: | aaggtgtgctaccggcCCCTCCtggctcaatctcc |
| 51): | ttgtctataggctgctg |

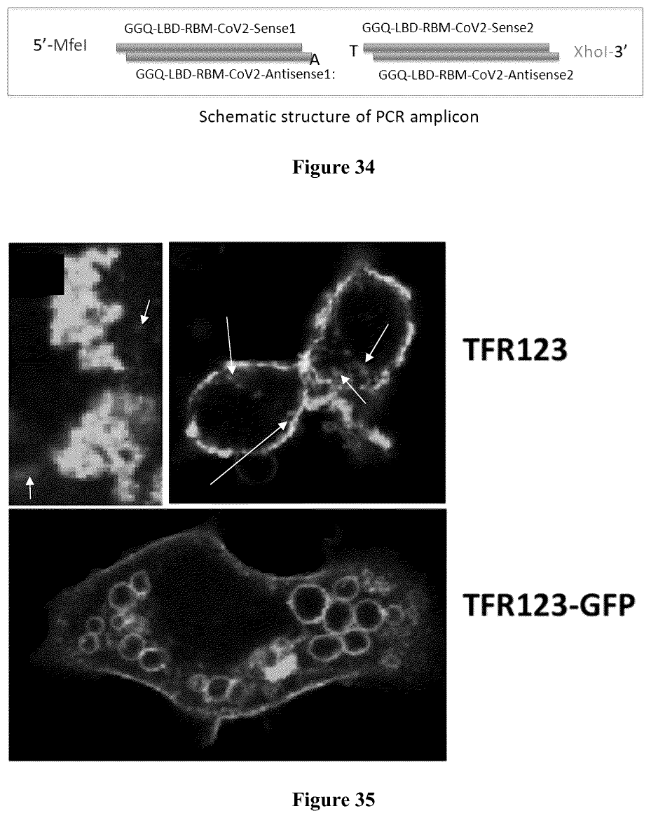

For sequences of Hybridization strategy see table 18. For the schematic structure of the PCR amplicon see FIG. 34.

Table 18. Nucleotide sequences for TFR123-A475-C488-pET28a construct (RBM): The table contains the nucleotide sequences hybridized to each other (Sense-1 to Antisense1, or Sense-2 to antisense-2). Hybridized double stranded DNAs having compatible ends to each other (-T and -A overhangs) as well as to the destination vector TFR123-pET28a digested with MfeIXhoI.

| Name of oligo | Nucleotide sequence 5′ to 3′ |
|---|---|
| GGQ-LBD-RBM-CoV2-Sense1 | aattgaccagcagcctatagacaaggagattgagc |
| (SEQ ID NO: 82): | caGGAGGGgccgg MfeI site |
| GGQ-LBD-RBM-CoV2-Antisense1 | accggcCCCTCCtggctcaatctccttgtctata |
| (SEQ ID NO: 83): | ggctgctggtc |
| GGQ-LBD-RBM-CoV2-Sense2 | tagcacaccttgtaatggtgttgaaggttttaattgtT |
| (SEQ ID NO: 84): | AAc |
| GGQ-LBD-RBM-CoV2-Antisense2 | tcgagTTAacaattaaaaccttcaacaccattaca |
| (SEQ ID NO: 85): | aggtgtgct |

### IV. Purification of TFR123-SARS-CoV-2 Monomers

Each DNA vector construct has been transformed into Escherichia coli BL21 protein expression strain. Transformation was performed via standard heat shock protocol. 100 mg vector insert construct was introduced to 100 ml of competent E. coli BL21 strain suspension on ice, and incubated it for 10 minutes on ice. The tubes were placed in a 42° C. water bath for exactly 1 minute, then incubate for 5 minutes on ice. 350 SOC medium was added to the suspension, and incubated for 90 minutes on 37° C. with 140 rpm in a shaker block. 5 and 100 ml of incubated suspension was spread on a 20 ml LB-agar plate containing 20 μl of Kanamycin (50 mg/ml). 10 ml of culture was started from the grown single colonies in 10 ml LB medium containing 10 μl Kanamycin. incubated overnight on 37° C. with 140 rpm. The 100 ml grown culture was added to 1000 ml of fresh LB containing 1 ml of Kanamycin, separated to two 500 ml glass container. The culture was incubated for 4 hours until the solution reached OD600, then 1 mM IPTG (Isopropyl β-D-1-thiogalactopyranoside, Isopropyl β-D-thiogalactoside) was added to the system to induce the desired protein expression. Following another 4 hours of incubation, the bacterial suspension had been centrifuged at 8000 rpm on 4° C. for 15 minutes and the pellets were frozen on −20° C. The pellets were thawed on ice in 20 ml Lysis buffer (300 mM NaCl; 50 mM NaH2PO4:Na2HPO4 1:1; 10 mM Imidazole; 0.5% Triton X, 0.5% Nonident NP-40; Protease inhibitor cocktail pH=8). The cells were lysed by 5×1 minute of ultrasound sonication with 50% pulse rate on ice, then centrifuged at 8000 rpm on 4° C. for 15 minutes. The supernatant was incubated with Nickel affinity chromatography beads overnight whilst shaking gently. The suspension was transferred to chromatography columns, and let rest until the beads descend completely. After the flow through were collected in a clean tube and the beads were washed 3×10 ml of excess lysis buffer then 3×10 ml of Washing buffer (300 mM NaCl, 50 mM NaH2PO4:Na2HPO4 1:1, Protease inhibitor cocktail pH=8.0). The expressed proteins were eluted vie Elution buffer (300 mM NaCl, 50 mM NaH2PO4:Na2HPO4 1:1, 1M Imidazole) 10×1 ml collected in separate microfuge tubes. During the process we collected SDS-PAGE samples from the steps indicated in square brackets. Concentration of the eluted proteins was identified by a BSA dilution series using densitometry assay. Recombinant peptide was dialyzed against refolding buffer (20 mM Tris pH=7.5, 150 mM NaCl, 10% glycerol, 3M urea). Recombinant peptide was sterile filtered with 0.22 μm MILLEX GP membrane.

### V. Assembly of TFR123-SARS-CoV-2 Monomers

Liposomes and purified TFR123-SARS-CoV-2 peptide are mixed in 1:1 weight ratio: 0.5 mg liposome to 0.5 mg recombinant peptide in refolding buffer (20 mM Tris pH=7.5, 150 mM NaCl, 10% glycerol, 3M urea). Assembly of oligomers will be induced by stepwise removal of urea. Liposome-peptide mixture will be dialyzed against refolding buffer contains 2M urea followed by 1.5M urea, 1M urea, 0.5M urea and no urea.

### VI. Immunization of Rabbits

For the immunization, we use 3-month-old rabbits. The rabbits receive an immunizing shot of 0.5 mg antigen, extended to 1.2 ml. The combined shots are extended with 400 μl CFL. Each rabbit receives 4×300 ml of subcutaneous injection in the neck area. Following the initial step, the animals receive a 0.25 mg boosting shot after two weeks, and another 0.25 mg shot one more week later. Rabbits will be bled two weeks later. Blood will be centrifuge and rabbit sera will be stored on ice and analysed by serological investigations (see VIII below).

### VII. Quantification of Immune Response of Rabbits

a. Determination of proinflammatory cytokines: To quantify immune response of rabbits injected with appropriate antigens, we intend to measure the changes of pro-inflammatory cytokines (TNF-alpha, IL2, IL6 etc.) by validated inflammatory ELISA kit. Very low immune response is expected by rabbits injected with TFR123 core peptide. However, TFR123-CoV-2-epitope extended immunogens (III.B.ii-iv and III.c.i-iv) might give remarkable immune response.

b. Detection of Immunoglobulin titters of sera after each boost of immunization:

IgM and IgG titter of diluted sera will be qualified by ELISA.

### VIII. Serological Investigation of the Sensitivity of Rabbit Sera

a. ELISA with immobilized recombinant full-length Spike protein In order to investigate and determine the sensitivity of rabbit sera, we intend to test serial diluted rabbit sera. Recombinant, purified full length Spike protein will be immobilized on ELISA plates and incubated with serial diluted rabbit sera (1:1, 1:2, 1:4, 1:8, 1:16 and 1:32).

b. ELISA with immobilized recombinant peptides used for immunization: The appropriate antigens (TFR123-SARS-CoV-2) used for immunization, will be immobilized on ELISA plate. The same experiment will be performed as above (VIII.a) c. Immunocytochemistry of HEK293 human embryonic kidney cells infected with SARS-CoV-2 using sera of immunized rabbits.

Infected HEK293 cells will be fixed with 4% PFA, permeabilised with digitonin, and blocked in 10% donkey sera. Primary antibody will be the serum of immunized rabbits. Secondary antibody will be anti-rabbit-AlexaFluor-488 fluorescens antibody. Fluorescent photographs will be taken by confocal microscope.

### IX. Serological Investigation of the Sensitivity of Human Sera Taken from Individuals Infected with SARS-CoV-2 and Having No Symptoms or Who Had Been Already Healed

To prove our concept, that outer loop of protease binding motif (PBM=TFR123-P807-R815) of SARS-CoV-2 Spike is a potential best epitope sequence for vaccination, we intend to test human sera taken from individuals infected with SARS-CoV-2 virus. The appropriate antigens (TFR123-SARS-CoV-2) used for immunization, will be immobilized on ELISA plate and incubated with serial diluted human sera (1:1, 1:2, 1:4, 1:8, 1:16 and 1:32)

### X. Investigation of the Effectivity of TFR123-CoV-2 Vaccine in Hamster

a. Investigation of immune response of control TFR123 core and TFR123-CoV-2 vaccines in hamster: To quantify immune response of hamster injected with appropriate antigens, we intend to measure the changes of pro-inflammatory cytokines (TNF-alpha, IL2, IL6 etc.) by validated inflammatory ELISA kit. Detection of Immunoglobulin titters of sera after each boost of immunization: IgM and IgG titter of diluted sera will be qualified by ELISA.

b. Quantification of virus copy number in hamster infected with SARS-CoV-2.

c. Quantification of virus copy number in hamster injected with TFR123-CoV2 thereafter infected with SARS-CoV2.

Results of immunization of rabbits with TFR123-S2-C13

Anti-SARS-CoV-2 antibodies from rabbits vaccinated with TFR123-S2-C13 showed a similar immunopositive reaction with the recombinant spike protein and total viral lysate as anti-SARS-CoV-2 antibodies from humans recovering from the infection with SARS-CoV-2 or anti-SARS-CoV-2 antibodies from humans vaccinated with Comirnaty.

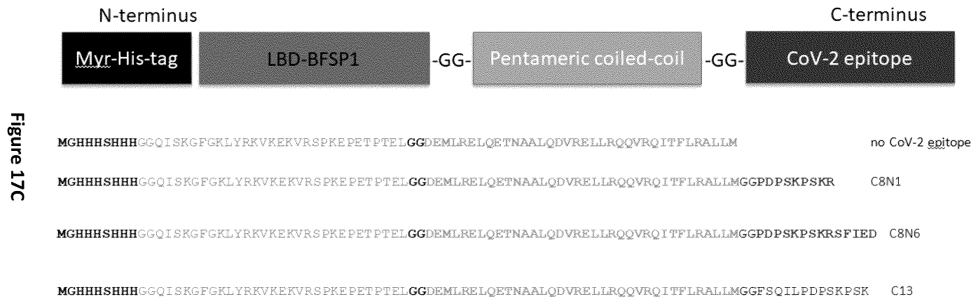

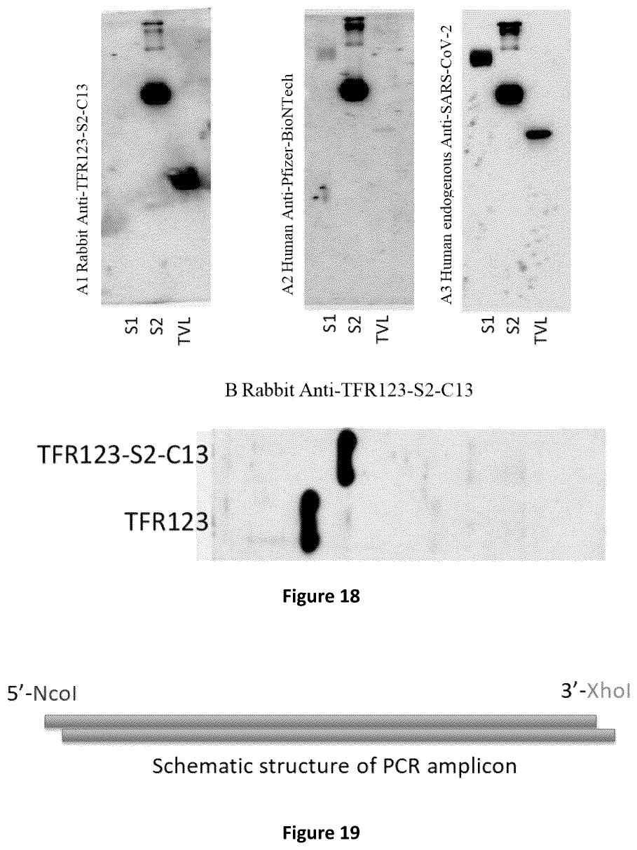

To show the specifity of the immune response induced by TFR123-S2-C13, we used blood samples from immunized rabbits drawn on the second week following the third vaccination with TFR123-S2-C13 (i.e, six weeks after the first shot). As a positive control, a serum sample from a subject vaccinated with Comirnaty (four weeks after the second shot) was used. Purified, recombinant SARS-CoV-2-S1, SARS-CoV-2-S2 and TVL (Total Viral lysate, i.e, inactivated lysate from Vero-6 cells transfected with SARS-CoV-2) served as antigens. The serum sample from the subject vaccinated with Comirnaty gave an immunopositive signal with both SARS-CoV-2-S1 and SARS-CoV-2-S2 (FIG. 18). Rabbit anti-TFR123-S2-C13 antibodies reacted immunopositively with recombinant SARS-CoV-2-S2 and a proteolytic fragment of the endogenous SARS-CoV-2-S2 protein (FIG. 18). Antibodies from the serum sample from a human recovering from the infection with SARS-CoV-2 showed immunopositivity with SARS-CoV-2-S1 and -S2, and proteolytic fragment of the endogenous SARS-CoV-2 spike protein (FIG. 18). Thus, polyclonal antibodies generated by the TFR123-S2-C13 vaccine are specific and sensitive for SARS-CoV-2.

### Tables

Table 19. PCR primers for TFR123 immune platform conjugated with CoV-2 epitopes

| Name of PCR primer | Sequences 5′ to 3′ |
|---|---|
| H3SH3-FOR (SEQ ID | ccATGGGCcaccaccacagccaccacc |
| NO: 30): | acggagggcagataagcaaaggc |
| NOHis REV (SEQ ID | CTCGAGTTAtggctcaatctccttgtc |
| NO: 31): | tatagg |
| C8-N1-REV (SEQ ID | ctcgagTTAcctcttgcttggttttgat |
| NO: 32): | ggatctggCCCTCCtggctcaatctcct |
| | tgtctatag |
| C8-N6-REV (SEQ ID | ctcgagTTAatcttcaataaatgacctc |
| NO: 33): | ttgcttggttttgatggatctggCCCTCC |
| | tggctcaatctccttgtctatag |
| C13-REV (SEQ ID | ctcgagTTActtgcttggttttgatggat |
| NO: 34): | ctggtaatatttgtgaaaaCCCTCCtggc |
| | tcaatctccttgtctatag |

Table 20. PCR primers for TER-3x3 immune platform conjugated with CoV-2 epitopes

| Name of | |
|---|---|
| oligonucleotide | PCR oligonucleotide: 5′-3′ |
| H3SH3For (SEQ | ccATGGGCcaccaccacagccaccaccacggagggcagataagcaaaggc |
| ID NO: 30) | |
| Rev-SacI (SEQ | gagctcagtgggggtctcagg |
| ID NO: 35) | |
| Rev-Trimer (SEQ | ctcgagttaagcacgctcgagttcttccagacg |
| ID NO: 36) | |
| For-Trimer (SEQ | gagctcggtggtcgtctgctagctcgtctggaag |
| ID NO: 37) | |
| Rev-Trimer- | ctcgagttacctcttgcttggttttgatggatctggccctccagcacgct |
| C8N1 (SEQ ID | cgagttcttccagacg |
| NO: 38) | |
| Rev-Trimer- | ctcgagttaatcttcaataaatgacctcttgcttggttttgatggatctg |
| C8N6 (SEQ ID | gccctccagcacgctcgagttcttccagacg |
| NO: 39) | |
| Rev-Ttrimer- | ctcgagTTActtgcttggttttgatggatctggtaatatttgtgaaaaCC |
| C13(SEQ ID NO: | CTCCagcacgctcgagttcttccagacg |
| 40) | |

Table 21. PCR primers for TFR-5x5 immune platform conjugated with CoV-2 epitopes

| Name of | |
|---|---|
| oligonucleotide | PCR oligonucleotide: 5′-3′ |
| H3SH3For (SEQ | ccATGGGCcaccaccacagccaccaccacggagggcagataagcaaaggc |
| ID NO: 30) | |
| Rev-SacI (SEQ | gagctcagtgggggtctcagg |
| ID NO: 35) | |
| Rev-Penta (SEQ | ctcgagttacatcagcagagcgcgcagaaag |
| ID NO: 41) | |
| For-Penta (SEQ | gagctcggaggggatgaaatgttgcgtgaattac |
| ID NO: 42) | |
| Rev-Penta-C8N1 | ctcgagttacctcttgcttggttttgatggatctggccctcccatcagca |
| (SEQ ID NO: 43) | gagcgcgcagaaag |
| Rev-Penta-C8N6 | ctcgagttaatcttcaataaatgacctcttgcttggttttgatggatctg |
| (SEQ ID NO: 44) | gccctcccatcagcagagcgcgcagaaag |
| Rev-Penta-C13 | ctcgagTTActtgcttggttttgatggatctggtaatatttgtgaaaaCC |
| (SEQ ID NO: 45) | CTCCtggctcaatctccttgtctatag |

Table 22. Examples of amino acid sequences of LBD variants

| SEQ | | |
|---|---|---|
| ID | | |
| NO | Name | AA sequence |
| 1 | hBFSP1 LBD short G434-T460 | GGQISKGFGKLYRKVKEKVRSPKEPET |
| 3 | hBFSP1 LBD long G434-E463 | GGQISKGFGKLYRKVKEKVRSPKEPETPTE |
| 130 | hBFSP1 LBD extra | GGQISKGFGKLYRKVKEKVRSPKEPETPTEL |

Table 23. Examples of amino acid sequences of OD variants

| SEQ | | |
|---|---|---|
| ID | | |
| NO | Name | AA sequence |
| 2 | hBFSP1 OD long | LYTKERHVLVTGDANYVDP |
| | L464-P548 | RFYVSSITAKGGVAVSVAE |
| | | DSVLYDGQVEPSPESPKPP |
| | | LENGQVGLQEKEDGQPIDQ |
| | | QPIDKEIEP |
| 120 | Trimeric OD | RLLARLEELERRLEELERR |
| | | LEELERA |
| 121 | Trimeric OD 2 | RLLARLEELERRLEELERR |
| | | LEELEAR |
| 122 | Pentameric OD | DEMLRELQETNAALQDVRE |
| | | LLRQQVRQITFLRALLM |
| 123 | short OD from | LYTKERHVLVTGDANYVDP |
| | filensin | RFYVSSITAKG |
| 124 | OD32 from filensin | LYTKERHVLVTGDANYVDP |
| | | RFYVSSITAKGGV |
| 125 | short trimer OD | LLARLEELERRLEELERRL |
| | | EELE |

### Immunization of Rabbits

The following groups were formed:
1. Rabbits receving Myr-GHHHSHHH-LBD-OD
2. Rabbits receving Myr-GHHHSHHH-LBD-OD-S2-C13
3. Rabbits receving Myr-GHHHSHHH-LBD-OD-RBM-C14
4. Rabbits receving Myr-GHHHSHHH-LBD-OD-RBD-C14
5. Rabbits receving Myr-GHHHSHHH-LBD-OD-RBD-N14
6. Rabbits receving Myr-GHHHSHHH-LBD-OD-S1S2-C14
7. Rabbits receving a combined vaccine of: Myr-LBD-OD-HHHHHH: GHHHSHHH-LBD-OD-S2-C13; GHHHSHHH-LBD-OD-RBM-C14: GHHHSHHH-LBD-OD-RBD-C14; GHHHSHHH-LBD-OD-RBD-N14; GHHHSHHH-LBD-OD-SIS2-C14

Specific anti-Spike-IgG antibodies are tested by ELISA and Western blot.

1-26. (canceled)

27. A recombinant peptide comprising a lipid binding amino acid sequence (LBD) derived from filensin (BFSP1), or a functional variant or functional fragment thereof capable of binding to a lipid.

28. The recombinant peptide according to claim 27, wherein a linker sequence GG is attached to the LBD.

29. The recombinant peptide according to claim 27, wherein the LBD comprises an amino acid sequence selected from the sequences according to SEQ ID NOs: 1, 3 and 130 and functional variants and functional fragments thereof capable of binding to a lipid, wherein the functional variant or functional fragment has a sequence with up to 10 amino acid difference from SEQ ID NO: 1, 3 or 130, respectively.

30. The recombinant peptide according to claim 27, wherein the LBD comprises the aminos acid sequence according to SEQ ID NO: 3 or a functional variant or a functional fragment thereof capable of binding to a lipid, wherein the functional variant or functional fragment has a sequence with up to 8 amino acid difference from SEQ ID NO: 3.

31. The recombinant peptide according to claim 27, further comprising an oligomerization amino acid sequence (OD).

32. The recombinant peptide according to claim 31, wherein the OD is derived from the BFSP1.

33. The recombinant peptide according to claim 31, wherein the OD comprises any one of the sequences according to SEQ ID NO: 2, SEQ ID NOs: 123-124 and functional variants and functional fragments thereof capable of oligomerization, wherein the functional variant or functional fragment has a sequence that is at least 85%, identical with SEQ ID NO: 2, SEQ ID NO: 123 or SEQ ID NO: 124, respectively.

34. The recombinant peptide according to claim 27, further comprising a bioactive agent.

35. The recombinant peptide according to claim 34, wherein the bioactive agent is an immunogenic agent (IM).

36. The recombinant peptide according to claim 35, wherein the IM is derived from SARS-CoV-2.

37. A method for the prevention or the treatment of a disease or a disorder, comprising administering a peptide comprising a lipid binding amino acid sequence (LBD) derived from filensin (BFSP1), or a functional variant or functional fragment thereof capable of binding to a lipid or the recombinant peptide according to claim 1 to a subject in need thereof.

38. The method according to claim 37, wherein a linker sequence GG is attached to the LBD comprised in the peptide or the recombinant peptide.

39. The method according to claim 37, wherein the LBD comprises an amino acid sequence selected from the sequences according to SEQ ID NOs: 1, 3 and 130 and functional variants and functional fragments thereof capable of binding to a lipid, wherein the functional variant or functional fragment has a sequence with up to 10 amino acid difference from SEQ ID NO: 1, 3 or 130, respectively.

40. The method according to claim 37, wherein the LBD comprises the aminos acid sequence according to SEQ ID NO: 3 or a functional variant or a functional fragment thereof capable of binding to a lipid, wherein the functional variant or functional fragment has a sequence with up to 8 amino acid difference from SEQ ID NO: 3.

41. The method according to claim 37, further comprising an oligomerization amino acid sequence (OD).

42. The method according to claim 41, wherein the OD is derived from the BFSP1.

43. The method according to claim 41, wherein the OD comprises any one of the sequences according to SEQ ID NO: 2, SEQ ID NOs: 123-124 and functional variants and functional fragments thereof capable of oligomerization, wherein the functional variant or functional fragment has a sequence that is at least 85%, identical with SEQ ID NO: 2, SEQ ID NO: 123 or SEQ ID NO: 124, respectively.

44. The method according to claim 37, further comprising a bioactive agent.

45. The method according to claim 44, wherein the bioactive agent is an immunogenic agent (IM).

46. The method according to claim 45, wherein the IM is derived from SARS-CoV-2.


SEQUENCE LISTING (130 sequences)

SEQ ID NO: 1 - protein, 27 aa, Homo sapiens
GGQISKGFGKLYRKVKEKVRSPKEPET

SEQ ID NO: 2 - protein, 85 aa, Homo sapiens
LYTKERHVLVTGDANYVDPRFYVSSITAKGGVAVSVAEDSVLYDGQVEPSPESPKPPLENGQVGLQEKEDGQPIDQQPIDKEIEP

SEQ ID NO: 3 - protein, 30 aa, Homo sapiens
GGQISKGFGKLYRKVKEKVRSPKEPETPTE

SEQ ID NO: 4 - protein, 665 aa, Homo sapiens
MYRRSYVFQTRKEQYEHADEASRAAEPERPADEGWAGATSLAALQGLGERVAAHVQRARALEQRHAGLRRQLDAFQRLGELAGPEDALARQVESNRQRVRDLEAERARLERQGTEAQRALDEFRSKYENECECQLLLKEMLERLNKEADEALLHNLRLQLEAQFLQDDISAAKDRHKKNLLEVQTYISILQQIIHTTPPASIVTSGMREEKLLTEREVAALRSQLEEGREVLSHLQAQRVELQAQTTTLEQAIKSAHECYDDEIQLYNEQIETLRKEIEETERVLEKSSYDCRQLAVAQQTLKNELDRYHRIIEIEGNRLTSAFIETPIPLFTQSHGVSLSTGSGGKDLTRALQDITAAKPRQKALPKNVPRRKEIITKDKTNGALEDAPLKGLEDTKLVQVVLKEESESKFESESKEVSPLTQEGAPEDVPDGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPDGAELEGPEEKREGEERDEESRRPCAMVTPGAEEPSIPEPPKPAADQDGAEVLGTRSRSLPEKGPPKALAYKTVEVVESIEKISTESIQTYEETAVIVETMIGKTKSDKKKSGEKSS

SEQ ID NO: 5 - protein, 15 aa, SARS coronavirus 2
  C8N6
LPDPSKPSKRSFIED

SEQ ID NO: 6 - protein, 14 aa, SARS coronavirus 2
  S2C14
FSQILPDPSKPSKR

SEQ ID NO: 7 - protein, 10 aa, SARS coronavirus 2
  C8N1
LPDPSKPSKR

SEQ ID NO: 8 - protein, 26 aa, Homo sapiens
  COMP OD 1
RLLARLEELERRLEELERRLEELERA

SEQ ID NO: 9 - protein, 36 aa, Homo sapiens
  COMP OD 2
DEMLRELQETNAALQDVRELLRQQVRQITFLRALLM

SEQ ID NO: 10 - protein, 9 aa, SARS coronavirus 2
LPDPSKPSK

SEQ ID NO: 11 - protein, 13 aa, SARS coronavirus 2
FSQILPDPSKPSK

SEQ ID NO: 12 - protein, 1273 aa, SARS coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 14 aa, SARS coronavirus 2
  S1S2C14
ASYQTQTNSPRRAR

SEQ ID NO: 14 - protein, 24 aa, SARS coronavirus 2
  S1S2C24
CDIPIGAGICASYQTQTNSPRRAR

SEQ ID NO: 15 - protein, 14 aa, SARS coronavirus 2
  RBD-N14
LKPFERDISTEIYQ

SEQ ID NO: 16 - protein, 14 aa, SARS coronavirus 2
  RBM-C14
AGSTPCNGVEGFNC

SEQ ID NO: 17 - protein, 14 aa, SARS coronavirus 2
  RBD-C14
YFPLQSYGFQPTNG

SEQ ID NO: 18 - protein, 24 aa, SARS coronavirus 2
  RBM-24
TEIYQAGSTPCNGVEGFNCYFPLQ

SEQ ID NO: 19 - protein, 24 aa, SARS coronavirus 2
  RBD-24
YFPLQSYGFQPTNGVGYQPYRVVV

SEQ ID NO: 20 - protein, 24 aa, SARS coronavirus 2
  S2-24
PPIKDFGGFNFSQILPDPSKPSKR

SEQ ID NO: 21 - protein, 14 aa, SARS coronavirus 2
  NUL-14
SKVGGNYNYLYRLF

SEQ ID NO: 22 - protein, 24 aa, SARS coronavirus 2
  NUL-24
VIAWNSNNLDSKVGGNYNYLYRLF

SEQ ID NO: 23 - protein, 14 aa, SARS coronavirus 2
  TCE1-C14
AYYVGYLQPRTFLL

SEQ ID NO: 24 - protein, 14 aa, SARS coronavirus 2
  TCE2-C14
SFSTFKCYGVSPTK

SEQ ID NO: 25 - protein, 14 aa, SARS coronavirus 2
  TCE3-C14
LGKYEQYIKWPWYI

SEQ ID NO: 26 - protein, 24 aa, SARS coronavirus 2
  TCE1-24
SSSGWTAGAAAYYVGYLQPRTFLL

SEQ ID NO: 27 - protein, 24 aa, SARS coronavirus 2
  TCE2-24
ADYSVLYNSASFSTFKCYGVSPTK

SEQ ID NO: 28 - protein, 24 aa, SARS coronavirus 2
  TCE3-24
LNESLIDLQELGKYEQYIKWPWYI

SEQ ID NO: 29 - protein, 70 aa, SARS coronavirus 2
  RBM-70
NSNNLDSKVGGNYNYLYRLFRKSNLKPFERDISTEIYQAGSTPCNGVEGFNCYFPLQSYGFQPTNGVGYQ

SEQ ID NO: 30 - DNA, 50 bp, Artificial Sequence
  primer
ccatgggccaccaccacagccaccaccacggagggcagataagcaaaggc

SEQ ID NO: 31 - DNA, 33 bp, Artificial Sequence
  primer
ctcgagttatggctcaatctccttgtctatagg

SEQ ID NO: 32 - DNA, 65 bp, Artificial Sequence
  primer
ctcgagttacctcttgcttggttttgatggatctggccctcctggctcaatctccttgtctatag

SEQ ID NO: 33 - DNA, 80 bp, Artificial Sequence
  primer
ctcgagttaatcttcaataaatgacctcttgcttggttttgatggatctggccctcctggctcaatctccttgtctatag

SEQ ID NO: 34 - DNA, 77 bp, Artificial Sequence
  primer
ctcgagttacttgcttggttttgatggatctggtaatatttgtgaaaaccctcctggctcaatctccttgtctatag

SEQ ID NO: 35 - DNA, 21 bp, Artificial Sequence
  primer
gagctcagtgggggtctcagg

SEQ ID NO: 36 - DNA, 33 bp, Artificial Sequence
  primer
ctcgagttaagcacgctcgagttcttccagacg

SEQ ID NO: 37 - DNA, 34 bp, Artificial Sequence
  primer
gagctcggtggtcgtctgctagctcgtctggaag

SEQ ID NO: 38 - DNA, 66 bp, Artificial Sequence
  primer
ctcgagttacctcttgcttggttttgatggatctggccctccagcacgctcgagttcttccagacg

SEQ ID NO: 39 - DNA, 81 bp, Artificial Sequence
  primer
ctcgagttaatcttcaataaatgacctcttgcttggttttgatggatctggccctccagcacgctcgagttcttccagacg

SEQ ID NO: 40 - DNA, 78 bp, Artificial Sequence
  primer
ctcgagttacttgcttggttttgatggatctggtaatatttgtgaaaaccctccagcacgctcgagttcttccagacg

SEQ ID NO: 41 - DNA, 31 bp, Artificial Sequence
  primer
ctcgagttacatcagcagagcgcgcagaaag

SEQ ID NO: 42 - DNA, 34 bp, Artificial Sequence
  primer
gagctcggaggggatgaaatgttgcgtgaattac

SEQ ID NO: 43 - DNA, 64 bp, Artificial Sequence
  primer
ctcgagttacctcttgcttggttttgatggatctggccctcccatcagcagagcgcgcagaaag

SEQ ID NO: 44 - DNA, 79 bp, Artificial Sequence
  primer
ctcgagttaatcttcaataaatgacctcttgcttggttttgatggatctggccctcccatcagcagagcgcgcagaaag

SEQ ID NO: 45 - DNA, 77 bp, Artificial Sequence; identical to SEQ ID NO: 34
  primer

SEQ ID NO: 46 - DNA, 59 bp, Artificial Sequence
  primer
ccatgggccaccaccaccaccaccacgatgtgccagatggagggcagataagcaaaggc

SEQ ID NO: 47 - DNA, 33 bp, Artificial Sequence; identical to SEQ ID NO: 31
  primer

SEQ ID NO: 48 - DNA, 81 bp, Artificial Sequence
  primer
ctcgagttacactttgttgaaaagtagatcttcaataaatgacctccctcctggctcaatctccttgtctataggctgctg

SEQ ID NO: 49 - DNA, 87 bp, Artificial Sequence
  primer
ctcgagttaaattttgccaatagcactattaaattggttggcaatcaatttccctcctggctcaatctccttgtctataggctgctg

SEQ ID NO: 50 - DNA, 90 bp, Artificial Sequence
  primer
ctcgagttaattgaggcggtcaatttctttttgaatgtttacaactgaagcattccctcctggctcaatctccttgtctataggctgctg

SEQ ID NO: 51 - DNA, 87 bp, Artificial Sequence
  primer
ctcgagttaacaattaaaaccttcaacaccattacaaggtgtgctaccggcccctcctggctcaatctccttgtctataggctgctg

SEQ ID NO: 52 - DNA, 26 bp, Artificial Sequence
  primer
ccatgggagggcagataagcaaaggc

SEQ ID NO: 53 - DNA, 30 bp, Artificial Sequence
  primer
ctcgagtggctcaatctccttgtctatagg

SEQ ID NO: 54 - DNA, 70 bp, Artificial Sequence
  oligo
catgggccaccaccaccaccaccacgatgtgccagatggagggcagataagcaaaggctttgggaaacta

SEQ ID NO: 55 - DNA, 65 bp, Artificial Sequence
  oligo
agtttcccaaagcctttgcttatctgccctccatctggcacatcgtggtggtggtggtggtggcc

SEQ ID NO: 56 - DNA, 59 bp, Artificial Sequence
  oligo
tacaggaaggtcaaggagaaagtgagaagccccaaagagcctgagacccccactgagct

SEQ ID NO: 57 - DNA, 56 bp, Artificial Sequence
  oligo
cagtgggggtctcaggctctttggggcttctcactttctccttgaccttcctgtat

SEQ ID NO: 58 - DNA, 46 bp, Artificial Sequence
  oligo
aattgaccagcagcctatagacaaggagattgagccaggagggcca

SEQ ID NO: 59 - DNA, 41 bp, Artificial Sequence
  oligo
ggccctcctggctcaatctccttgtctataggctgctggtc

SEQ ID NO: 60 - DNA, 28 bp, Artificial Sequence
  oligo
gatccatcaaaaccaagcaagaggtaac

SEQ ID NO: 61 - DNA, 33 bp, Artificial Sequence
  oligo
tcgagttacctcttgcttggttttgatggatct

SEQ ID NO: 62 - DNA, 43 bp, Artificial Sequence
  oligo
gatccatcaaaaccaagcaagaggtcatttattgaagattaac

SEQ ID NO: 63 - DNA, 48 bp, Artificial Sequence
  oligo
tcgagttaatcttcaataaatgacctcttgcttggttttgatggatct

SEQ ID NO: 64 - DNA, 49 bp, Artificial Sequence
  oligo
aattgaccagcagcctatagacaaggagattgagccaggagggttttca

SEQ ID NO: 65 - DNA, 44 bp, Artificial Sequence
  oligo
gaaaaccctcctggctcaatctccttgtctataggctgctggtc

SEQ ID NO: 66 - DNA, 40 bp, Artificial Sequence
  oligo
caaatattaccagatccatcaaaaccaagcaagaggtaac

SEQ ID NO: 67 - DNA, 45 bp, Artificial Sequence
  oligo
tcgagttacctcttgcttggttttgatggatctggtaatatttgt

SEQ ID NO: 68 - DNA, 44 bp, Artificial Sequence
  oligo
aattgaccagcagcctatagacaaggagattgagccaggaggga

SEQ ID NO: 69 - DNA, 39 bp, Artificial Sequence
  oligo
ccctcctggctcaatctccttgtctataggctgctggtc

SEQ ID NO: 70 - DNA, 39 bp, Artificial Sequence
  oligo
ggtcatttattgaagatctacttttcaacaaagtgtaac

SEQ ID NO: 71 - DNA, 44 bp, Artificial Sequence
  oligo
tcgagttacactttgttgaaaagtagatcttcaataaatgacct

SEQ ID NO: 72 - DNA, 46 bp, Artificial Sequence; identical to SEQ ID NO: 58
  oligo

SEQ ID NO: 73 - DNA, 41 bp, Artificial Sequence; identical to SEQ ID NO: 59
  oligo

SEQ ID NO: 74 - DNA, 28 bp, Artificial Sequence
  oligo
gatccatcaaaaccaagcaagaggtaac

SEQ ID NO: 75 - DNA, 33 bp, Artificial Sequence; identical to SEQ ID NO: 61
  oligo

SEQ ID NO: 76 - DNA, 49 bp, Artificial Sequence
  oligo
aattgaccagcagcctatagacaaggagattgagccaggagggaatgct

SEQ ID NO: 77 - DNA, 44 bp, Artificial Sequence
  oligo
gcattccctcctggctcaatctccttgtctataggctgctggtc

SEQ ID NO: 78 - DNA, 43 bp, Artificial Sequence
  oligo
tcagttgtaaacattcaaaaagaaattgaccgcctcaattaac

SEQ ID NO: 79 - DNA, 48 bp, Artificial Sequence
  oligo
tcgagttaattgaggcggtcaatttctttttgaatgtttacaactgaa

SEQ ID NO: 80 - DNA, 48 bp, Artificial Sequence
  oligo
aattgaccagcagcctatagacaaggagattgagccaggaggggccgg

SEQ ID NO: 81 - DNA, 45 bp, Artificial Sequence
  oligo
accggcccctcctggctcaatctccttgtctataggctgctggtc

SEQ ID NO: 82 - DNA, 41 bp, Artificial Sequence
  oligo
tagcacaccttgtaatggtgttgaaggttttaattgttaac

SEQ ID NO: 83 - DNA, 44 bp, Artificial Sequence
  oligo
tcgagttaacaattaaaaccttcaacaccattacaaggtgtgct

SEQ ID NO: 84 - DNA, 378 bp, Artificial Sequence
  pET-28a-c(+) cloning region
agatctcgatcccgcgaaattaatacgactcactataggggaattgtgagcggataacaattcccctctagaaataattttgtttaactttaagaaggagatataccatgggcagcagccatcatcatcatcacagcagcggcctggtgccgcgcggcagccatatggctagcatgactggtggacagcaaatgggtcgcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 85 - DNA, 472 bp, Artificial Sequence
  pET-28b(+) cloning region
agatctcgatcccgcgaaattaatacgactcactataggggaattgtgagcggataacaattcccctctagaaataattttgtttaactttaagaaggagatataccatgggcagcagccatcatcatcatcacagcagcggcctggtgccgcgcggcagccatatggctagcatgactggtggacagcaaatgggtcgcggatccgaattcgagctccgtcgacaagcttgcggccgcactcgagcaccaccaccaccaccactgagatccggctgctaacaaagcccggtcgggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 86 - DNA, 471 bp, Artificial Sequence
  pET-28c(+) cloning region
agatctcgatcccgcgaaattaatacgactcactataggggaattgtgagcggataacaattcccctctagaaataattttgtttaactttaagaaggagatataccatgggcagcagccatcatcatcatcacagcagcggcctggtgccgcgcggcagccatatggctagcatgactggtggacagcaaatgggtcgcggatccgaattcgagctccgtcgacaagcttgcggccgcactcgagcaccaccaccaccaccactgagatccggctgctaacaaagcccggtcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 87 - protein, 58 aa, Artificial Sequence
  pET-28a(+) expression region
MGSSHHHHHHSSGLVPRGSHMASMTGGQQMGRGSEFELRRQACGRTRAPPPPPLRSGC

SEQ ID NO: 88 - protein, 52 aa, Artificial Sequence
  pET-28b(+) expression region
MGSSHHHHHHSSGLVPRGSHMASMTGGQQGRDPNSSSVDKLAAALEHHHHHH

SEQ ID NO: 89 - protein, 60 aa, Artificial Sequence
  pET-28c(+) expression region
MGSSHHHHHHSSGLVPRGSHMASMTGGQQGRIRIRAPSTSLRPHSSTTTTTTEIRLLTKP

SEQ ID NO: 90 - protein, 22 aa, SARS coronavirus 2
FSQILPDPSKPSKRSFIEDLLF

SEQ ID NO: 91 - protein, 127 aa, Artificial Sequence
  His-tag+caspase site+TFR123 platform and no epitope
MGHHHHHHDVPDGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEP

SEQ ID NO: 92 - protein, 138 aa, Artificial Sequence
  His-tag+caspase site+TFR123 platform and C8N1
MGHHHHHHDVPDGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGGPDPSKPSKR

SEQ ID NO: 93 - protein, 143 aa, Artificial Sequence
  His-tag+caspase site+TFR123 platform and C8N6
MGHHHHHHDVPDGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGGPDPSKPSKRSFIED

SEQ ID NO: 94 - protein, 142 aa, Artificial Sequence
  His-tag+caspase site+TFR123 platform and C13
MGHHHHHHDVPDGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGGFSQILPDPSKPSK

SEQ ID NO: 95 - protein, 124 aa, Artificial Sequence
  Myr-His-tag+TFR123+linker no epitope
MGHHHSHHHGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEP

SEQ ID NO: 96 - protein, 135 aa, Artificial Sequence
  Myr-His-tag+FR123+ linker + SARS-CoV-2 C8N1 epitope
MGHHHSHHHGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGGPDPSKPSKR

SEQ ID NO: 97 - protein, 140 aa, Artificial Sequence
  Myr-His-tag+TFR123+linker + SARS-CoV-2 C8N6 epitope
MGHHHSHHHGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGGPDPSKPSKRSFIED

SEQ ID NO: 98 - protein, 139 aa, Artificial Sequence
  Myr-His-tag+TFR123+linker + SARS-CoV-2 C13 epitope
MGHHHSHHHGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGGFSQILPDPSKPSK

SEQ ID NO: 99 - protein, 68 aa, Artificial Sequence
  Myr-His-tag+TFR3x3 platform and no epitope
MGHHHSHHHGGQISKGFGKLYRKVKEKVRSPKEPETPTELGGRLLARLEELERRLEELERRLEELERA

SEQ ID NO: 100 - protein, 79 aa, Artificial Sequence
  Myr-His-tag+TFR3x3 platform and C8N1
MGHHHSHHHGGQISKGFGKLYRKVKEKVRSPKEPETPTELGGRLLARLEELERRLEELERRLEELERAGGPDPSKPSKR

SEQ ID NO: 101 - protein, 84 aa, Artificial Sequence
  Myr-His-tag+TFR3x3 platform and C8N6
MGHHHSHHHGGQISKGFGKLYRKVKEKVRSPKEPETPTELGGRLLARLEELERRLEELERRLEELERAGGPDPSKPSKRSFIED

SEQ ID NO: 102 - protein, 83 aa, Artificial Sequence
  Myr-His-tag+TFR3x3 platform and C13
MGHHHSHHHGGQISKGFGKLYRKVKEKVRSPKEPETPTELGGRLLARLEELERRLEELERRLEELERAGGFSQILPDPSKPSK

SEQ ID NO: 103 - protein, 78 aa, Artificial Sequence
  Myr-His-tag+TFR5x5 platform and no epitope
MGHHHSHHHGGQISKGFGKLYRKVKEKVRSPKEPETPTELGGDEMLRELQETNAALQDVRELLRQQVRQITFLRALLM

SEQ ID NO: 104 - protein, 89 aa, Artificial Sequence
  Myr-His-tag+TFR5x5 platform and C8N1
MGHHHSHHHGGQISKGFGKLYRKVKEKVRSPKEPETPTELGGDEMLRELQETNAALQDVRELLRQQVRQITFLRALLMGGPDPSKPSKR

SEQ ID NO: 105 - protein, 94 aa, Artificial Sequence
  Myr-His-tag+TFR5x5 platform and C8N6
MGHHHSHHHGGQISKGFGKLYRKVKEKVRSPKEPETPTELGGDEMLRELQETNAALQDVRELLRQQVRQITFLRALLMGGPDPSKPSKRSFIED

SEQ ID NO: 106 - protein, 93 aa, Artificial Sequence
  Myr-His-tag+TFR5x5 platform and C13
MGHHHSHHHGGQISKGFGKLYRKVKEKVRSPKEPETPTELGGDEMLRELQETNAALQDVRELLRQQVRQITFLRALLMGGFSQILPDPSKPSK

SEQ ID NO: 107 - protein, 9 aa, Artificial Sequence
  Mye-His tag
MGHHHSHHH

SEQ ID NO: 108 - protein, 123 aa, Artificial Sequence
  TFR123 platform variant A
GGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPLEHHHHHH

SEQ ID NO: 109 - protein, 125 aa, Artificial Sequence
  TFR123 variant B
GHHHSHHHGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGG

SEQ ID NO: 110 - protein, 126 aa, Artificial Sequence
  TFR123 variant C
GHHHSKHHHGGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGG

SEQ ID NO: 111 - protein, 125 aa, Artificial Sequence
  TFR123 variant D
GGQISKGFGKLYRKVKEKVRSPKEPETPTELYTKERHVLVTGDANYVDPRFYVSSITAKGGVAVSVAEDSVLYDGQVEPSPESPKPPLENGQVGLQEKEDGQPIDQQPIDKEIEPGGHHHHHHGG

SEQ ID NO: 112 - protein, 125 aa, Artificial Sequence; identical to SEQ ID NO: 109
  TFR123 variant F

SEQ ID NO: 113 - DNA, 380 bp, Artificial Sequence
  DNA sequence of TFR123 -Myr-His-tag
ccatgggccaccaccacagccaccaccacggagggcagataagcaaaggctttgggaaactatacaggaaggtcaaggagaaagtgagaagccccaaagagcctgagacccccactgagctctacaccaaagagcggcacgtgctggtcacaggggatgccaattacgtggaccctagattctatgtctcctccatcacagctaaaggtggggtggctgtttctgttgcggaagactctgtgctttatgacggccaggtggagccctctcctgagtcacccaagccccctttagagaatgggcaggtgggtctgcaggagaaagaagatggacaaccaattgaccagcagcctatagacaaggagattgagccaggaggg

SEQ ID NO: 114 - DNA, 383 bp, Artificial Sequence
  DNA sequence of TFR123 Myr-His-tag-extra
ccatgggccaccaccacagcaaacaccaccacggagggcagataagcaaaggctttgggaaactatacaggaaggtcaaggagaaagtgagaagccccaaagagcctgagacccccactgagctctacaccaaagagcggcacgtgctggtcacaggggatgccaattacgtggaccctagattctatgtctcctccatcacagctaaaggtggggtggctgtttctgttgcggaagactctgtgctttatgacggccaggtggagccctctcctgagtcacccaagccccctttagagaatgggcaggtgggtctgcaggagaaagaagatggacaaccaattgaccagcagcctatagacaaggagattgagccaggaggg

SEQ ID NO: 115 - DNA, 374 bp, Artificial Sequence
  DNA sequence of TFR123 inter-chain His-tag
ccatgggagggcagataagcaaaggctttgggaaactatacaggaaggtcaaggagaaagtgagaagccccaaagagcctgagacccccactgagctctacaccaaagagcggcacgtgctggtcacaggggatgccaattacgtggaccctagattctatgtctcctccatcacagctaaaggtggggtggctgtttctgttgcggaagactctgtgctttatgacggccaggtggagccctctcctgagtcacccaagccccctttagagaatgggcaggtgggtctgcaggagaaagaagatggacaaccaattgaccagcagcctatagacaaggagattgagccaggagggcaccaccaccaccaccac

SEQ ID NO: 116 - DNA, 378 bp, Artificial Sequence
  DNA sequence of HHHHHH-TFR123
ccatggtcaccaccaccaccaccacgagggcagataagcaaaggctttgggaaactatacaggaaggtcaaggagaaagtgagaagccccaaagagcctgagacccccactgagctctacaccaaagagcggcacgtgctggtcacaggggatgccaattacgtggaccctagattctatgtctcctccatcacagctaaaggtggggtggctgtttctgttgcggaagactctgtgctttatgacggccaggtggagccctctcctgagtcacccaagccccctttagagaatgggcaggtgggtctgcaggagaaagaagatggacaaccaattgaccagcagcctatagacaaggagattgagccataactcgag

SEQ ID NO: 117 - DNA, 376 bp, Artificial Sequence
  DNA sequence of TFR123-HHHHHH
ccatggagggcagataagcaaaggctttgggaaactatacaggaaggtcaaggagaaagtgagaagccccaaagagcctgagacccccactgagctctacaccaaagagcggcacgtgctggtcacaggggatgccaattacgtggaccctagattctatgtctcctccatcacagctaaaggtggggtggctgtttctgttgcggaagactctgtgctttatgacggccaggtggagccctctcctgagtcacccaagccccctttagagaatgggcaggtgggtctgcaggagaaagaagatggacaaccaattgaccagcagcctatagacaaggagattgagccactcgagcaccaccaccaccaccactga

SEQ ID NO: 118 - RNA, 353 nt, Artificial Sequence
  RNA sequence of TFR123
  modified_base 2: /mod_base=“m1f”
  modified_base 2: /mod_base=“m1f”
  modified_base 13: /mod_base=“m1f”
  modified_base 13: /mod_base=“m1f”
  modified_base 24: /mod_base=“m1f”
  modified_base 24: /mod_base=“m1f”
  modified_base 25: /mod_base=“m1f”
  modified_base 25: /mod_base=“m1f”
  modified_base 26: /mod_base=“m1f”
  modified_base 26: /mod_base=“m1f”
  modified_base 34: /mod_base=“m1f”
  modified_base 34: /mod_base=“m1f”
  modified_base 36: /mod_base=“m1f”
  modified_base 36: /mod_base=“m1f”
  modified_base 46: /mod_base=“m1f”
  modified_base 46: /mod_base=“m1f”
  modified_base 58: /mod_base=“m1f”
  modified_base 77: /mod_base=“m1f”
  modified_base 89: /mod_base=“m1f”
  modified_base 94: /mod_base=“m1f”
  modified_base 96: /mod_base=“m1f”
  modified_base 115: /mod_base=“m1f”
  modified_base 118: /mod_base=“m1f”
  misc_feature 120: /note=“n is 1-methyl-3′-pseudouridylyl”
  modified_base 121: /mod_base=“m1f”
  modified_base 131: /mod_base=“m1f”
  modified_base 137: /mod_base=“m1f”
  modified_base 138: /mod_base=“m1f”
  modified_base 142: /mod_base=“m1f”
  modified_base 149: /mod_base=“m1f”
  modified_base 153: /mod_base=“m1f”
  modified_base 154: /mod_base=“m1f”
  modified_base 156: /mod_base=“m1f”
  modified_base 158: /mod_base=“m1f”
  modified_base 160: /mod_base=“m1f”
  modified_base 162: /mod_base=“m1f”
  modified_base 165: /mod_base=“m1f”
  modified_base 169: /mod_base=“m1f”
  modified_base 176: /mod_base=“m1f”
  modified_base 182: /mod_base=“m1f”
  modified_base 187: /mod_base=“m1f”
  modified_base 191: /mod_base=“m1f”
  modified_base 193: /mod_base=“m1f”
  modified_base 193: /mod_base=“m1f”
  modified_base 194: /mod_base=“m1f”
  modified_base 195: /mod_base=“m1f”
  modified_base 197: /mod_base=“m1f”
  modified_base 199: /mod_base=“m1f”
  modified_base 200: /mod_base=“m1f”
  modified_base 210: /mod_base=“m1f”
  modified_base 212: /mod_base=“m1f”
  modified_base 214: /mod_base=“m1f”
  modified_base 217: /mod_base=“m1f”
  modified_base 218: /mod_base=“m1f”
  modified_base 219: /mod_base=“m1f”
  modified_base 221: /mod_base=“m1f”
  modified_base 232: /mod_base=“m1f”
  modified_base 240: /mod_base=“m1f”
  modified_base 242: /mod_base=“m1f”
  modified_base 245: /mod_base=“m1f”
  modified_base 249: /mod_base=“m1f”
  modified_base 263: /mod_base=“m1f”
  modified_base 264: /mod_base=“m1f”
  modified_base 265: /mod_base=“m1f”
  modified_base 272: /mod_base=“m1f”
  modified_base 280: /mod_base=“m1f”
  modified_base 284: /mod_base=“m1f”
  modified_base 286: /mod_base=“m1f”
  modified_base 302: /mod_base=“m1f”
  modified_base 313: /mod_base=“m1f”
  modified_base 314: /mod_base=“m1f”
  modified_base 326: /mod_base=“m1f”
  modified_base 328: /mod_base=“m1f”
  modified_base 340: /mod_base=“m1f”
  modified_base 341: /mod_base=“m1f”
anggagggcaganaagcaaaggcnnngggaaacnanacaggaaggncaaggagaaagngagaagccccaaagagccngagacccccacngagcncnacaccaaagagcggcacgngcnggncacaggggangccaannacgnggacccnaganncnangncnccnccancacagcnaaaggnggggnggcngnnncngnngcggaagacncngngcnnnangacggccaggnggagcccncnccngagncacccaagcccccnnnagagaangggcaggngggncngcaggagaaagaaganggacaaccaanngaccagcagccnanagacaaggaganngagccaggaggg

SEQ ID NO: 119 - RNA, 409 nt, Artificial Sequence
  UTR and poly(A) tail to TFR123 RNA
  modified_base 8: /mod_base=“m1f”
  modified_base 14: /mod_base=“m1f”
  modified_base 17: /mod_base=“m1f”
  modified_base 20: /mod_base=“m1f”
  modified_base 24: /mod_base=“m1f”
  modified_base 33: /mod_base=“m1f”
  modified_base 36: /mod_base=“m1f”
  modified_base 40: /mod_base=“m1f”
  modified_base 46: /mod_base=“m1f”
  modified_base 47: /mod_base=“m1f”
  modified_base 48: /mod_base=“m1f”
  modified_base 53: /mod_base=“m1f”
  modified_base 56: /mod_base=“m1f”
  modified_base 60: /mod_base=“m1f”
  modified_base 69: /mod_base=“m1f”
  modified_base 71: /mod_base=“m1f”
  modified_base 81: /mod_base=“m1f”
  modified_base 86: /mod_base=“m1f”
  modified_base 93: /mod_base=“m1f”
  modified_base 95: /mod_base=“m1f”
  modified_base 95: /mod_base=“m1f”
  modified_base 98: /mod_base=“m1f”
  modified_base 105: /mod_base=“m1f”
  modified_base 111: /mod_base=“m1f”
  modified_base 119: /mod_base=“m1f”
  modified_base 127: /mod_base=“m1f”
  modified_base 129: /mod_base=“m1f”
  modified_base 132: /mod_base=“m1f”
  modified_base 135: /mod_base=“m1f”
  modified_base 136: /mod_base=“m1f”
  modified_base 146: /mod_base=“m1f”
  modified_base 163: /mod_base=“m1f”
  modified_base 169: /mod_base=“m1f”
  modified_base 178: /mod_base=“m1f”
  modified_base 179: /mod_base=“m1f”
  modified_base 184: /mod_base=“m1f”
  modified_base 211: /mod_base=“m1f”
  modified_base 214: /mod_base=“m1f”
  modified_base 215: /mod_base=“m1f”
  modified_base 220: /mod_base=“m1f”
  modified_base 221: /mod_base=“m1f”
  modified_base 222: /mod_base=“m1f”
  modified_base 228: /mod_base=“m1f”
  modified_base 238: /mod_base=“m1f”
  modified_base 239: /mod_base=“m1f”
  modified_base 240: /mod_base=“m1f”
  modified_base 244: /mod_base=“m1f”
  modified_base 249: /mod_base=“m1f”
  modified_base 251: /mod_base=“m1f”
  modified_base 254: /mod_base=“m1f”
  modified_base 264: /mod_base=“m1f”
  modified_base 265: /mod_base=“m1f”
  modified_base 268: /mod_base=“m1f”
  modified_base 272: /mod_base=“m1f”
  modified_base 273: /mod_base=“m1f”
  modified_base 274: /mod_base=“m1f”
  modified_base 277: /mod_base=“m1f”
  modified_base 291: /mod_base=“m1f”
  modified_base 297: /mod_base=“m1f”
  modified_base 334: /mod_base=“m1f”
  modified_base 336: /mod_base=“m1f”
  modified_base 340: /mod_base=“m1f”
ugaugacncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaccccagggnnggncaannncgngccagccacacccnggagcnagcaaaaaaaaaaaaaaaaaaaaaaaaaaaaaagcanangacnaaaaaaaaaaaaaaaaaaaaaaaaaaaaaaaaaaaaaaaaaaaaaaaaaaaaaaaaaaaaaaaaaaaaa

SEQ ID NO: 120 - protein, 26 aa, Artificial Sequence
  Trimeric OD
RLLARLEELERRLEELERRLEELERA

SEQ ID NO: 121 - protein, 26 aa, Artificial Sequence
  Trimeric OD 2
RLLARLEELERRLEELERRLEELEAR

SEQ ID NO: 122 - protein, 36 aa, Artificial Sequence; identical to SEQ ID NO: 9
  Pentameric OD

SEQ ID NO: 123 - protein, 30 aa, Homo sapiens
  short OD from filensin
LYTKERHVLVTGDANYVDPRFYVSSITAKG

SEQ ID NO: 124 - protein, 32 aa, Homo sapiens
  OD32 from filensin
LYTKERHVLVTGDANYVDPRFYVSSITAKGGV

SEQ ID NO: 125 - protein, 23 aa, Artificial Sequence
  short trimer OD
LLARLEELERRLEELERRLEELE

SEQ ID NO: 126 - DNA, 345 bp, Artificial Sequence
  DNA sequence of TFR123
ggagggcagataagcaaaggctttgggaaactatacaggaaggtcaaggagaaagtgagaagccccaaagagcctgagacccccactgagctctacaccaaagagcggcacgtgctggtcacaggggatgccaattacgtggaccctagattctatgtctcctccatcacagctaaaggtggggtggctgtttctgttgcggaagactctgtgctttatgacggccaggtggagccctctcctgagtcacccaagccccctttagagaatgggcaggtgggtctgcaggagaaagaagatggacaaccaattgaccagcagcctatagacaaggagattgagcca

SEQ ID NO: 127 - DNA, 108 bp, Artificial Sequence
  DNA sequence of the pentamer OD
gatgaaatgttgcgtgaattacaggaaaccaacgctgctctgcaagacgttcgtgaactgctgcgtcaacaggttcgtcagatcacctttctgcgcgctctgctgatg

SEQ ID NO: 128 - DNA, 72 bp, Artificial Sequence
  DNA sequence of the trimeric OD
cgtctgctagctcgtctggaagaactggaaagacgtctggaagaactggaacgtcgtctggaagaactcgag

SEQ ID NO: 129 - DNA, 133 bp, Artificial Sequence
  DNA sequence or the long trimeric OD
cgtctgctagctcgtctggaagaactggaaagacgtctggaagaactggaacgtcgtctggaagaactcgagcgtgctatcaacaccgtcgacctggaactggctgctctgcgtcgtcgtctggaagaactgg

SEQ ID NO: 130 - protein, 31 aa, Artificial Sequence
  hBFSP1 LBD extra
GGQISKGFGKLYRKVKEKVRSPKEPETPTEL